(19) United States
(12) Patent Application Publication
(10) Pub. No.: US 2021/0228729 A1
(43) Pub. Date: Jul. 29, 2021

(54) LGALS3BP ANTIBODY-DRUG-CONJUGATE AND ITS USE FOR THE TREATMENT OF CANCER

(71) Applicant: MEDIAPHARMA S.R.L., Chieti (IT)

(72) Inventors: Emily CAPONE, Cavallino (IT); Francesco GIANSANTI, Scoppito (IT); Rodolfo IPPOLITI, Rome (IT); Roberta GENTILE, L'Aquila (IT); Stefano IACOBELLI, Rome (IT); Enza PICCOLO, Lanciano (IT); Sara PONZIANI, Penna Sant'Andrea (IT); Gianluca SALA, Pescara (IT)

(21) Appl. No.: 17/046,832

(22) PCT Filed: Apr. 12, 2019
(86) PCT No.: PCT/EP2019/059505

§ 371 Date: Oct. 12, 2020

(30) Foreign Application Priority Data
Apr. 12, 2018 (EP) 18166980.5

(51) Int. Cl. A61K 47/68; A61K 31/5365; A61P 35/04; A61K 39/00

(52) U.S. Cl. CPC A61K 47/6803; A61K 31/5365; A61K 2039/505; A61K 47/6849; A61P 35/04

(57) ABSTRACT

The present invention relates to a special type of non-internalizing binding moiety-drug-conjugates that specifically target LGALS3BP. From one aspect, the invention relates to an antibody-drug-conjugate comprising an antibody capable of binding to LGALS3BP, said antibody being conjugated to cytotoxic drugs. The invention also comprises methods of the treatment of LGALS3BP-expressing cancer, including administering to a patient the disclosed drug conjugates and pharmaceutical preparations.

### FIELD OF THE INVENTION

The present invention relates to a binding moiety-drug-conjugate capable of binding LGALS3BP. From one aspect, the invention relates to a antibody-drug-conjugate comprising a non-internalizing antibody capable of binding to LGALS3BP, said antibody being conjugated to cytotoxic drugs. The invention also comprises methods of treatment and the use of said antibody-drug-conjugate for the treatment of LGALS3BP-expressing cancer.

### BACKGROUND OF THE INVENTION

The use of cytotoxic agents is at the basis of the medical treatment of cancer and other pathological conditions. Although these agents preferentially accumulate at the tumor sites, a certain amount reaches healthy organs, causing cytotoxic side effects. One possible solution to avoid or limit the lack of selectivity of cytotoxic agents is to couple these agents to an antibody to form an Antibody-Drug Conjugate (ADC) recognizing specifically a target antigen expressed at the cell surface that is unique to or expressed at higher levels in cancer cells types. Unfortunately, several technical difficulties have been encountered with the ADC approach. A first drawback with the ADC approach is that the targets have been limited to targets that internalize upon ADC binding. In some cases, even though the target for the ADC exists on the cell surface, internalization does not occur. This makes the ADC approach cell type specific and target specific. Complicating this even further are the cases where the target is expressed and internalization occurs, but the internalization is within compartments where drug antibody dissociation does not occur, leaving the drug ineffective.

Another difficulty encountered with the ADC approach relates to how much active drug can be delivered inside the cell. Generally, there are only a small number of copies of each different disease-specific antigen binding site at the cell surface, and the number of drug molecules that can be linked to a single antibody without interfering with antigen (target) binding is relatively low (between 2 to 10 with a mean of 4 per antibody). These two factors in combination have made the ADC approach practical only when very potent (typically very toxic) drugs are used.

Given all these constraints, it is not surprising that only a few ADCs for application in oncology: Gemtuzumab ozogamicin (Mylotarg®), brentuximab vedotin (Adcetris®), trastuzumab emtansine (Kadcyla™), and Inotuzumab ozogamicin (Besponsa®) have been available on the market. Therefore, there continues to be a need for improved ADCs that circumvent these requirements and/or overcome the difficulties and drawbacks of existing methods.

More recently, a type of ADCs which do not need to be internalized by cancer cells in order to kill or inhibit them has been investigated. These non-internalizing ADCs target antigens that are structural components of the stroma (environment) surrounding tumor cells. Examples are those based on antibodies targeting fibronectin splicing variants containing extra domain A and antibodies targeting tenascin C (REFS).

The human Lectin Galactoside-Binding Soluble 3-Binding Protein (LGALS3BP) is the target antigen of the present invention. A murine anti-LGALS3BP (also known as 90K) previously described (WO2010/097825A1), has been humanized and named 1959. In this invention, the antibody has been further engineered by substitution of 3 cysteines by 3 serines. The resulting ADC has the capacity to deliver sufficient cytotoxic drug to the target cells, providing an innovative and effective treatment for cancer.

LGALS3BP, also known as 90K and Mac-2 binding protein is a heavily glycosylated, large oligomeric, human protein consisting of about 90 kDa subunits. The protein was originally isolated from the conditioned medium of human breast cancer cells (1).

Functionally, LGALS3BP has been shown to mediate adhesive processes, including homotypic cell adhesion and cell to extra-cellular matrix (ECM) adhesion (2). Recently, the protein has been shown to stimulate tumor angiogenesis through a pathway independent of that used by Vascular Endothelial Growth Factor (VEGF) (3).

Several endogenous ligands of LGALS3BP have been identified, including galectins (4), integrins (5), tetraspanins (6), the ECM proteins collagens IV, V and VI, fibronectin and nidogen (7), endosialin (8) and CD33-related Siglecs (9). Some of these ligands are stable components of the plasma membrane of cancer cells. For example, interaction of the tetraspanin CD9/CD82 web with LGALS3BP has been reported to occur in colorectal cancer (10). Also, data have been reported to show interaction of LGALS3BP with membrane residues of galectin-3 and galectin-1 to promote homothypic aggregation of adjacent melanoma cells (11). Most of these interactions are finalized to tumor progression and metastasis formation (2).

LGALS3BP was found to be up-regulated in many human cancers, including breast carcinoma, non-small cell lung carcinoma, lymphoma, pleural mesothelioma, melanoma, pancreatic carcinoma, neuroblastoma (9) LGALS3BP is a secreted protein. Particularly, tumor cells produce and secrete elevated amounts of LGALS3BP. Elevated levels of LGALS3BP, both in the serum and tumor tissues have been significantly associated with a shorter survival, the occurrence of metastasis or a reduced response to chemotherapy in patients affected by different types of malignancies (2). Most of the patients affected by LGALS3BP-expressing cancers have a bad prognosis.

The inhibitory effect of the anti-LGALS3BP antibody in the murine form called SP-2 or in the humanized variant called 1959 has been investigated. The focus has been on the use of unconjugated antibodies to neutralize the adhesive and the pro-angiogenic properties of LGALS3BP, thus inhibiting growth and progression of human cancer. However, one main shortcoming of such an approach was the only partial inhibitory effect on tumor growth (12). Thus, there is a need in the art for developing a more powerful approach for neutralizing the growth of LGALS3BP-expressing cancers.

### BRIEF SUMMARY OF THE INVENTION

The present invention relates to a special type of non-internalizing drug conjugate, in particular ADC that targets LGALS3BP, wherein such conjugate upon binding to tumor cells expressing LGALS3BP exerts a cytotoxic effect and kills the LGALS3BP-expressing tumor cells.

The present invention further provides methods for the treatment of a LGALS3BP-expressing cancer in a patient. The methods generally include administering to the patient an effective amount of the ADC includes an antibody that binds to LGALS3BP. The antibody is conjugated to a drug that is a cytotoxic agent and kills the tumor cells.

In certain embodiments, the antibody is a monoclonal antibody against LGALS3BP. In other embodiments, the heavy chain variable region has at least 80% sequence identity, preferably at least 90% sequence identity to the amino acid sequence set forth in SEQ ID NO:1, and the light chain variable region has at least 80% sequence identity, preferably at least 90% sequence identity to the amino acid sequence set forth in SEQ ID NO:2.

In another embodiment, the antibody is derived from the humanized antibody 1959, wherein the cysteines of the hinge region of the antibody 1959 at position 220, 226 and 229 are substituted by serines, to form the 1959-sss antibody.

In exemplary embodiments, the antibody-drug conjugates are 1959-sss-DM1, 1959-sss-DM3, and 1959-sss-DM4.

The present invention further provides a pharmaceutical composition comprising an ADC indicated above and a pharmaceutically acceptable carrier.

The present invention further provides a method of treating a LGALS3BP-expressing cancer in a patient in need thereof, comprising administering to said patient the ADC indicated above.

The present invention further provides a method of treating a LGALS3BP-expressing cancer wherein said cancer is selected from the group consisting of colorectal cancer, breast cancer, pancreatic cancer, non-small cell lung cancer, melanoma, glioblastoma, neuroblastoma, carcinomas, melanoma, glioblastoma, neuroblastoma and lymphoma.

The invention further provides an antibody-drug conjugate indicated above for use in therapy. The invention further provides the use of an antibody-drug conjugate indicated above for the manufacture of a medicament.

The invention further provides the use indicated above, wherein said use is for the treatment of a LGALS3BP-expressing cancer and wherein said cancer is selected from the group consisting of colorectal cancer, breast cancer, pancreatic cancer, non-small cell lung cancer, melanoma, glioblastoma, neuroblastoma, carcinomas, melanoma, glioblastoma, neuroblastoma and lymphoma.

The invention further provides a nucleic acid that encodes an anti-LGALS3BP antibody, wherein the cysteines of the hinge region of the humanized antibody 1959 at position 220, 226 and 229 are substituted by serines.

The invention further provides a process for producing an anti-LGALS3BP antibody-drug conjugate comprising: (a) conjugating a cytotoxic drug to an anti-LGALS3BP antibody recovered from cell culture, and; (c) purifying the antibody-drug conjugate.

In antibody drug conjugates, the antibody can be conjugated directly to the cytotoxic agent, or via a linker. Direct conjugation may occur via formation of a disulfide bond between SH-derivatized paylods and the cysteine residues of the antibody. Suitable linkers include, for example, cleavable and non-cleavable linkers, containing or not a disulfide bond.

In yet additional aspects, pharmaceutical compositions are provided for the treatment of a LGALS3BP-expressing cancer. The pharmaceutical compositions include an antibody-drug conjugate and at least one pharmaceutically compatible ingredient.

The present invention may be more fully understood by reference to the following detailed description of the invention, non-limiting examples of specific embodiments of the invention and the appended figures.

### BRIEF DESCRIPTION OF THE DRAWINGS

FIG. 1: Absorbance spectrum of the antibody 1959-sss after derivatization with DTNB.

FIG. 2: G25 column separation of the complex 1959-sss-DM3 from unreacted free DM3

FIG. 3: Absorbance spectrum of the antibody 1959-sss after the reaction with DM3

FIG. 4: Calibration curve construction by HPLC

FIG. 5: HPLC analysis of DM3 release from 1959-sss-DM3 conjugate following reduction with TCEP

FIG. 6: HIC-HPLC analysis of unconjugated 1959-sss and 1959-sss-DM3

FIG. 7: MALDI mass spectrometry analysis of naked 1959-sss (upper panel) and conjugated 1959-sss-DM3 (lower panel)

FIG. 8: light chains MALDI mass spectrometry analysis of naked 1959-sss (upper panel) and conjugated 1959-sss-DM3 (lower panel).

FIG. 9: MALDI mass spectrometry analysis of 1959-sss after reduction with TCEP

FIG. 10: HIC chromatography of 1959-sss-DM3 (upper panel), 1959-sss-DM3 after reduction with TCEP (middle panel), naked 1959-sss.

FIG. 11: Binding of unconjugated 1959-sss and 1959-sss-DM3 to LGALS3BP

FIG. 12: Expression of LGALS3BP in human tumor cell lines

FIG. 13: Co-localization of Gal-3BP and CD63 and CD81 at the membrane of human melanoma cells. Staining upon incubation with 1959-sss alone or in combination with CD 63 and CD81 followed by either anti-human fluorescently labelled IgG or anti-mouse fluorescently labelled IgG. LGALS3BP co-localizes with the exosome marker proteins at the cell membrane. Staining was granular, possibly indicating clustering induced by LGALS3BP upon secretion.

FIG. 14: In Vivo therapeutic efficacy of 1959-sss-DM1 and 1959-sss-DM3 in a xenograft model of melanoma

FIG. 15: In Vivo therapeutic efficacy of 1959-sss-DM3 and 1959-sss-DM4 in a xenograft model of melanoma

FIG. 16: Dose-response of 1959-sss-DM3 in a xenograft model of melanoma

FIG. 17: Evaluation of LGALS3BP (Gal-3BP) expression and secretion by neuroblastoma cells. (A) RT-PCR, (B) western blot and (C) ELISA.

FIG. 18: Membrane staining. 1959 antibody specifically stains LGALS3BP at the membrane of LGALS3BP positive but not negative neuroblastoma cells

FIG. 19: Activity of Maytansine-derivative SH-DM3 in neuroblastoma cells

FIG. 20: 1959-sss/DM3: therapeutic activity in neuroblastoma is target dependent

(A): SKNAS (Gal-3BP positive); ADC administration: 4 doses, twice-weekly, 10 mg/kg

(B): hNB (Gal-2BP negative); ADC administration: 4 doses, twice-weekly, 10 mg/kg

(C): in vivo 1959-sss/DM3 accumulation. SKNAS (Gal-3BP positive)

FIG. 21: 1959-sss/DM3: therapeutic activity in experimental metastasis neuroblastoma models

(A): Experimental metastasis assay

(B): assay in SKNAS neuroblastoma cell model

(C): assay in Kelly neuroblastoma cell model

FIG. 22: results of 1959-sss/DM3 pharmacokinetic studies in mouse serum

Humanized SP-2 variant was generated by identifying murine complementarity determining regions (CDRs) that were grafted onto a human antibody framework as described (13,14).

The amino acid sequences for the heavy chain and the light chain variable regions of murine SP2 antibody were determined by cDNA sequencing of the hybridoma cell line producing SP2 antibody. The complementarity determining regions (CDRs) for the VL and VH were identified and grafted into a human IgG framework. Amino acid sequence of humanized HC and LC 1959 antibody are defined as SEQ ID NO:9 and respectively. CDRs are underlined. The amino acid sequence of HC 1959-sss is defined as SEQ ID NO:11. CDRs are underlined and bold residues in italics represent Cysteine→Serine substitutions.

SEQ ID NO: 11
QVQLQESGPGLVKPSETLSLTCAVSGYSISSGYYWTWIRQPPGKGLEWIGY
ITYDGKNNYSPSLKNRVTISVDTSKNQFSLKLSSVTAADTAVYYCAREGSS
VITTGFTFWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYIC
NVNHKPSNTKVDKKVEPKS [symbol] DKTHT [symbol] PP [symbol] PAPELLGGPSVFLFPPKPK
DTLMISRTPEVT

### DESCRIPTION OF THE INVENTION

The methods and compositions described herein encompass the use of non-internalizing ADC's or derivatives that (a) specifically target LGALS3BP and (b) kill LGALS3BP-expressing cancer cells. As used herein, the term “derivative,” in the context of an anti-LGALS3BP antibody, refers to a molecule that (i) has an antigen-binding region of an anti-LGALS3BP antibody, or a region derived therefrom (e.g., by conservative substitution), and at least one polypeptide region or other moiety heterologous to the anti-LGALS3BP antibody, and (ii) specifically binds to LGALS3BP via the antigen-binding region or region derived therefrom. In specific embodiments, the anti-LGALS3BP antibody is antibody 1959 or 1959-sss or a derivative thereof.

In typical embodiments, the anti-LGALS3BP antibody or derivative thereof, when conjugated to a cytotoxic agent, kills LGALS3BP-expressing cancer cells.

Anti-LGALS3BP antibodies suitable for use in accordance with the present compositions and methods are typically monoclonal and can include, for example, chimeric (e.g., having a human constant region and mouse variable region), humanized, or human antibodies; single chain antibodies; or the like. The immunoglobulin molecules can be of any type (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments, the antibody is an antigen-binding antibody fragment such as, for example, a Fab, a F(ab′), a F(ab′)₂, a Fd chain, a single-chain Fv (scFv), a single-chain antibody, a disulfide-linked Fv (sdFv), a fragment comprising either a V_{L} or V_{H} domain, or fragments produced by a Fab expression library, or a LGALS3BP-binding fragments of any of the above antibodies described supra. Antigen-binding antibody fragments, including single-chain antibodies, can comprise the variable region(s) alone or in combination with the entirety or a portion of the following: hinge region, CH1, CH2, CH3 and CL domains. Also, antigen-binding fragments can comprise any combination of variable region(s) with a hinge region, CH1, CH2, CH3 and CL domains. Typically, the antibodies are human, rodent (e.g., mouse and rat), donkey, sheep, rabbit, goat, guinea pig, camelid, horse, or chicken. As used herein, “human” antibodies include antibodies having the amino acid sequence of a human immunoglobulin and include antibodies isolated from human immunoglobulin libraries, from human B cells, or from animals transgenic for one or more human immunoglobulin, as described infra and, for example in U.S. Pat. Nos. 5,939,598 and 6,111,166.

The antibodies may be monospecific, bispecific, trispecific, or of greater multispecificity. Multispecific antibodies may be specific for different epitopes of LGALS3BP or may be specific for both LGALS3BP as well as for a heterologous protein. (15, 16, 17, 18, 19, 20).

Techniques for generating antibody fragments that recognize specific epitopes are also generally known in the art. For example, Fab and F(ab′)₂ fragments can be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH 1 domain of the heavy chain. Techniques to recombinantly produce Fab, Fab′ and F(ab′)₂ fragments can also be employed using, e.g., methods disclosed in PCT publication (21, 22, 23, 24).

Examples of techniques that can be used to produce single-chain Fvs and antibodies include those described in 25, 26, 27, 28, 29.

In certain embodiments, the anti-LGALS3BP antibody is a chimeric antibody. A chimeric antibody is a molecule in which different portions of the antibody are derived from different animal species, such as for example antibodies having a variable region derived from a murine monoclonal antibody and a human immunoglobulin constant region. Methods for producing chimeric antibodies are known in the art. (30, 31, 32, 33, 34, 35).

An anti-LGALS3BP antibody can also be a humanized antibody. Humanized antibodies are antibody molecules that bind the desired antigen and have one or more CDRs from a non-human species, and framework and constant regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (36, 37). Antibodies can be humanized using a variety of techniques known in the art including, for example, CDR-grafting (38, 39, 40, 41, 42); veneering or resurfacing (43, 44, 45 46, 47), and chain shuffling (48).

In yet other embodiments, the anti-LGALS3BP antibody is a human antibody. Human antibodies can be made by a variety of methods known in the art including, e.g., phage display methods (see supra) using antibody libraries derived from human immunoglobulin sequences (49, 50, 51, 52, 53, 54, 55, 56, 57). In addition, a human antibody recognizing a selected epitope can be generated using a technique referred to as “guided selection,” in which a selected non-human monoclonal antibody, e.g., a mouse antibody, is used to guide the selection of a completely human antibody recognizing the same epitope (58). Human antibodies can also be produced using transgenic mice that express human immunoglobulin genes. Monoclonal antibodies directed against the antigen can be obtained from the immunized, transgenic mice using conventional hybridoma technology. For an overview of this technology for producing human antibodies, see ref. (59). For a detailed discussion of this technology for producing human antibodies and human monoclonal antibodies and protocols for producing such antibodies, see ref. 53, 60, 55, 56, 61, 62, 63, 64, 65, 66, 67, 68, 69, 70, 71. In addition, companies such as Abgenix, Inc. (Fremont, Calif.) and Medarex (Princeton, N.J.) can be engaged to provide human antibodies directed against a selected antigen using technology similar to that described above.

As set forth supra, a derivative of an anti-LGALS3BP antibody can also be used in the practice of present methods. Generally, an anti-LGALS3BP antibody derivative comprises an anti-LGALS3BP antibody (including, e.g., an antigen-binding fragment or conservatively substituted polypeptides) and at least one polypeptide region or other moiety heterologous to the anti-LGALS3BP antibody. For example, an anti-LGALS3BP antibody can be modified, e.g., by the covalent attachment of any type of molecule, such that covalent attachment does not prevent the antibody derivative from specifically binding to LGALS3BP via the antigen-binding region or region derived therefrom, or the conjugated drug from exerting (a) a cytostatic or cytotoxic effect on LGALS3BP-expressing cancer cells. Typical modifications include, e.g., glycosylation, acetylation, pegylation, phosphorylation, amidation, derivatization by known protecting/blocking groups, proteolytic cleavage, linkage to a cellular ligand or other protein, and the like. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

In certain embodiments, the antibody derivative is a multimer, such as, for example, a dimer, comprising one or more monomers, where each monomer includes (i) an antigen-binding region of an anti-LGALS3BP antibody, or a polypeptide region derived therefrom (such as, e.g., by conservative substitution of one or more amino acids), and (ii) a multimerizing (e.g., dimerizing) polypeptide region, such that the antibody derivative forms multimers (e.g., homodimers) that specifically bind to LGALS3BP. In typical embodiments, an antigen binding region of an anti-LGALS3BP antibody, or a polypeptide region derived therefrom, is recombinantly or chemically fused with a heterologous protein, wherein the heterologous protein comprises a dimerization or multimerization domain. Prior to administration of the antibody derivative to a subject for the purpose of treating or preventing LGALS3BP-expressing cancers, the derivative is subjected to conditions that allow formation of a homodimer or heterodimer. A heterodimer, as used herein, may comprise identical dimerization domains but different LGALS3BP antigen-binding regions, identical LGALS3BP antigen-binding regions but different dimerization domains, or different LGALS3BP antigen-binding regions and dimerization domains.

In yet other embodiments, the dimerization domain is an immunoglobulin constant region such as, for example, a heavy chain constant region or a domain thereof (e.g., a CH1 domain, a CH2 domain, or a CH3 domain). (72, 73, 74, 75, 76, 77)

In other embodiments, an anti-LGALS3BP antibody derivative is an anti-LGALS3BP antibody conjugated to a second antibody (an “antibody heteroconjugate”) (78). Heteroconjugates useful for practicing the present methods comprise an antibody that binds to LGALS3BP (e.g., an antibody that has the CDRs and/or heavy chains of the monoclonal antibody 1959 or 1959-sss and an antibody that binds to a surface receptor or receptor complex, such as an immunoglobulin gene superfamily member, a TNF receptor superfamily member, an integrin, a cytokine receptor, a chemokine receptor, a major histocompatibility protein, a lectin (C-type, S-type, or I-type), or a complement control protein.

Antibodies can be assayed for specific binding to LGALS3BP by any of various known methods. Immunoassays which can be used include, for example, competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well-known in the art. (79, 80).

According to a particularly preferred aspect of the present invention, the anti-LGALS3BP antibody is the antibody 1959. Accordingly, a preferred anti-LGALS3BP antibody comprises a heavy chain amino acid sequence as shown in SEQ ID NO: 9 and a light chain amino acid sequence as shown in SEQ ID NO: 10. Further, a preferred anti-LGALS3BP antibody comprises at least the heavy and light chain variable regions of 1959. The amino acid sequence of the heavy chain variable region is as shown in SEQ ID NO: 1 and the amino acid sequence of the light chain variable region is shown in SEQ ID NO: 2. In other embodiments, the anti-LGALS3BP antibody comprises a heavy chain variable region having at least 80% sequence identity, preferably at least 90% sequence identity to the amino acid sequence set forth in SEQ ID NO:1, and a light chain variable region having at least 80% sequence identity, preferably at least 90% sequence identity to the amino acid sequence set forth in SEQ ID NO:2. A further preferred anti-LGALS3BP antibody is characterised as comprising the six CDR sequences of the antibody 1959. The CDR sequences of the heavy chain are CDRH1 as shown in SEQ ID NO: 3, CDRH2 as shown in SEQ ID NO: 4 and CDRH3 as shown in SEQ ID NO: 5. The CDR sequences of the light chain are CDRL1 as shown in SEQ ID NO: 6, CDRL2 as shown in SEQ ID NO: 7 and CDRL3 as shown in SEQ ID NO: 8.

According to another preferred embodiment, the anti-LGALS3BP antibody is the antibody 1959-sss. In the antibody 1959-sss the cysteines of the hinge region of the humanized antibody 1959 at position 220, 226 and 229 are substituted by serines. The heavy chain amino acid sequence of 1959-sss is shown in SEQ ID NO: 11. Accordingly, a preferred anti-LGALS3BP antibody comprises a heavy chain amino acid sequence as shown in SEQ ID NO: 11 and a light chain amino acid sequence as shown in SEQ ID NO: 10.

In another embodiment, LGALS3BP once secreted from tumor cells remains in close proximity to the plasma membrane, where it may bind to several membrane-associated LGALS3BP endogenous ligands.

In additional embodiments, anti-LGALS3BP antibody or derivatives thereof can be targeted to tumor cells expressing and secreting LGALS3BP.

The anti-LGALS3BP antibodies and derivatives thereof that are useful in the present methods can be produced by any method known in the art for the synthesis of proteins, typically, e.g., by recombinant expression techniques. For example, for recombinant expression of an anti-LGALS3BP antibody, an expression vector may encode a heavy or light chain thereof, or a heavy or light chain variable domain, operably linked to a promoter. An expression vector may include, for example, the nucleotide sequence encoding the constant region of the antibody molecule (81, 82, 83), and the variable domain of the antibody may be cloned into such a vector for expression of the entire heavy or light chain. The expression vector is transferred to a host cell by conventional techniques, and the transfected cells are then cultured by conventional techniques to produce the anti-LGALS3BP antibody. In typical embodiments for the expression of double-chained antibodies, vectors encoding both the heavy and light chains can be co-expressed in the host cell for expression of the entire immunoglobulin molecule.

A variety of prokaryotic and eukaryotic host-expression vector systems can be utilized to express an anti-LGALS3BP antibody or derivative thereof. Typically, eukaryotic cells, particularly for whole recombinant anti-LGALS3BP antibody molecules, are used for the expression of the recombinant protein. For example, mammalian cells such as Chinese hamster ovary cells (CHO), in conjunction with a vector such as the major intermediate early gene promoter element from human cytomegalovirus, is an effective expression system for the production of anti-LGALS3BP antibodies and derivatives thereof (84, 85)

A stable expression system is typically used for long-term, high-yield production of recombinant anti-LGALS3BP antibody or derivative thereof. For example, cell lines that stably express the anti-LGALS3BP antibody or derivative thereof can be engineered by transformation of host cells with DNA controlled by appropriate expression control elements (e.g., promoter, enhancer, sequences, transcription terminators, polyadenylation sites) and a selectable marker, followed by growth of the transformed cells in a selective media. The selectable marker confers resistance to the selection and allows cells to stably integrate the DNA into their chromosomes and grow to form foci which in turn can be cloned and expanded into cell lines. A number of selection systems can be used, including, for example, the herpes simplex virus thymidine kinase, hypoxanthineguanine phosphoribosyltransferase, and adenine phosphoribosyltransferase genes, which can be employed in tk⁻, hgprt⁻ or aprt⁻ cells, respectively. Also, antimetabolite resistance can be used as the basis of selection for the following genes: dhfr, which confers resistance to methotrexate; gpt, which confers resistance to mycophenolic acid; neo, which confers resistance to the aminoglycoside G-418; and hygro, which confers resistance to hygromycin. Methods commonly known in the art of recombinant DNA technology can be routinely applied to select the desired recombinant clone, and such methods are described, for example, in 86, 87, 88, 89.

The expression levels of an antibody or derivative can be increased by vector amplification. (90). When a marker in the vector system expressing an anti-LGALS3BP antibody or derivative thereof is amplifiable, an increase in the level of inhibitor present in host cell culture media will select host cells that have increased copy number of a marker gene conferring resistance to the inhibitor. The copy number of an associated antibody gene will also be increased, thereby increasing expression of the antibody or derivative thereof (91).

Where the anti-LGALS3BP antibody comprises both a heavy and a light chain or derivatives thereof, the host cell may be co-transfected with two expression vectors, the first vector encoding the heavy chain protein and the second vector encoding the light chain protein. The two vectors may contain identical selectable markers which enable equal expression of heavy and light chain proteins. Alternatively, a single vector may be used which encodes, and is capable of expressing, both heavy and light chain proteins. In such situations, the light chain is typically placed before the heavy chain to avoid an excess of toxic free heavy chain (92, 93). The coding sequences for the heavy and light chains may comprise cDNA or genomic DNA.

Once an anti-LGALS3BP antibody or derivative thereof has been produced (e.g., by an animal, chemical synthesis, or recombinant expression), it can be purified by any suitable method for purification of proteins, including, for example, by chromatography (e.g., ion exchange or affinity chromatography (such as, for example, Protein A chromatography for purification of antibodies having an intact Fc region)), centrifugation, differential solubility, or by any other standard technique for the purification of proteins. An anti-LGALS3BP antibody or derivative thereof can, for example, be fused to a marker sequence, such as a peptide, to facilitate purification by affinity chromatography. Suitable marker amino acid sequences include, e.g., a hexa-histidine peptide, such as the tag provided in a pQE vector (QIAGEN, Inc., 9259 Eton Avenue, Chatsworth, Calif., 91311), and the “HA” tag, which corresponds to an epitope derived from the influenza hemagglutinin protein (94), and the “flag” tag.

### Construction of Anti-LGALS3Bp Antibody-Drug Conjugates

Compositions useful in the treatment of a LGALS3BP-expressing cancer comprise anti-LGALS3BP antibody-drug conjugates (ADCs) or anti-LGALS3BP ADC derivatives. An “anti-LGALS3BP ADC” as used herein refers to an anti-LGALS3BP antibody conjugated to a therapeutic agent. An “anti-LGALS3BP derivative ADC” as used herein refers to derivative of an anti-LGALS3BP antibody conjugated to a therapeutic agent. In certain embodiments, the ADC comprises an anti-LGALS3BP antibody (e.g., 1959 antibody or a fragment or derivative thereof). The ADCs or ADC derivatives as described herein produce clinically beneficial effects on LGALS3BP-expressing cells when administered to a patient with a LGALS3BP-expressing cancer, typically when administered alone but also in combination with other therapeutic agents.

In typical embodiments, the anti-LGALS3BP antibody or derivative thereof is conjugated to a cytotoxic agent, such that the resulting ADC or ADC derivative exerts a killing effect on a LGALS3BP expressing cancer cell. Particularly suitable moieties for conjugation to antibodies or antibody derivatives are chemotherapeutic agents, prodrug converting enzymes, radioactive isotopes or compounds, or toxins. For example, an anti-LGALS3BP antibody or derivative thereof can be conjugated to a cytotoxic agent such as a chemotherapeutic agent, or a toxin (e.g., a cytostatic or cytocidal agent such as, e.g., abrin, ricin A, pseudomonas exotoxin, or diphtheria toxin). Examples of additional agents that are useful for conjugating to the anti-LGALS3BP molecules are provided infra.

Techniques for conjugating therapeutic agents to proteins, and in particular to antibodies, are well-known. (95, 96, 97, 98, 99, 100).

In accordance with the methods described herein, the anti-LGALS3BP ADC or ADC derivative is not internalized, but rather accumulates extracellularly, at the surface of LGALS3BP-expressing cells.

In other embodiments, the anti-LGALS3BP ADC or ADC derivative is not internalized, and the therapeutic agent is effective to bind to LGALS3BP-expressing cell at the cell membrane. In yet other embodiments, because of the reducing environment of the extracellular environment, the drug is released from the ADC, then diffuses inside the cancer cell and kills it.

To maximize activity of the therapeutic agent outside the LGALS3BP-expressing cancer cells, the 1959 antibody that specifically binds to LGALS3BP can be used. As LGALS3BP is continuously secreted and remains in close proximity of cell membrane, the therapeutic agent is concentrated at the cell surface of LGALS3BP-expressing cancer cells. In a more typical embodiment, the therapeutic agent is conjugated in a manner that displays its activity when cleaved off the antibody by reduction of the disulfide bond. In such embodiments, the therapeutic agent is attached to the antibody with a disulfide bond that is sensitive to the reducing conditions of the tumor extracellular environment.

### Cytotoxic Agents

Suitable cytotoxic agents can be, for example, an auristatin, a DNA minor groove binding agent, a DNA minor groove alkylating agent, an enediyne, a lexitropsin, a duocarmycin, a taxane, a puromycin, a dolastatin, a maytansinoid, and a vinca alkaloid. In specific embodiments, the drug is cytotoxic agent is DM1, DM3, DM4, AFP, MMAF, MMAE, AEB, AEVB, auristatin E, paclitaxel, docetaxel, CC-1065, SN-38, topotecan, morpholino-doxorubicin, rhizoxin, cyanomorpholino-doxorubicin, dolastatin-10, echinomycin, combretatstatin, chalicheamicin, maytansine, DMb 1, or netropsin. Other suitable cytotoxic agents include anti-tubulin agents, such as an auristatin, a vinca alkaloid, a podophyllotoxin, a taxane, a baccatin derivative, a cryptophysin, a maytansinoid, a combretastatin, or a dolastatin. In specific embodiments, the antitubulin agent are the maytansinoids DM1, DM3, and DM4.

In certain embodiments, the therapeutic agent is a radioisotope, including ¹³¹Iodine.

### Other LGALS3Bp-Targeting Moieties and Protein-Drug Conjugates

As indicated supra, in other embodiments, the LGALS3BP-targeting moiety need not be an antibody to be useful in accordance with the methods described herein. Accordingly, a LGALS3BP-targeting moiety can include one or more CDRs from an antibody that binds to LGALS3BP and when conjugated to a cytotoxic agent kills the tumor cells. Typically, the protein is a multimer, most typically a dimer.

Further, LGALS3BP-binding proteins useful in accordance with the methods provided herein include fusion proteins, i.e., proteins that are recombinantly fused or chemically conjugated (including both covalent and non-covalent conjugation) to heterologous proteins (of typically at least 10, 20, 30, 40, 50, 60, 70, 80, 90 or at least 100 amino acids). The fusion does not necessarily need to be direct, but may occur through linker sequences.

For example, LGALS3BP-targeting moieties useful in the present methods can be produced recombinantly by fusing the coding region of one or more of the CDRs of an anti-LGALS3BP antibody in frame with a sequence coding for a heterologous protein. The heterologous protein may provide one or more of the following characteristics: added therapeutic benefits; promote stable expression; provide a means of facilitating high yield recombinant expression; and/or provide a multimerization domain.

In the context of the present invention, also such conjugates which do not contain an antibody, but another type of LGALS3BP binding moiety, are referred to as “ADC”. The term “ADC” therefore shall not be considered as limiting in that an antibody must be contained, but rather that also conjugates which can contain an antibody or any other binding moieties are comprised by said term, unless indicated otherwise.

### Pharmaceutical Compositions Comprising Anti-LGALS3Bp ADC and ADC Derivatives and Administration Thereof

In accordance with the present methods, a composition comprising an anti-LGALS3BP ADC or ADC derivative as described herein is administered to a subject having a LGALS3BP-expressing cancer. The term “subject” as used herein means any mammalian patient to which a LGALS3BP-binding protein-drug conjugate may be administered, including, e.g., humans and non-human mammals, such as primates, rodents, and dogs. Subjects specifically intended for treatment using the methods described herein include humans. The ADCs or ADC derivatives can be administered either alone or in combination with other compositions in the prevention or treatment of LGALS3BP-expressing cancer.

Various delivery systems are known and can be used to administer the anti-LGALS3BP ADC or ADC derivative. Methods of introduction include but are not limited to intradermal, intramuscular, intraperitoneal, intravenous, subcutaneous, intranasal, epidural, and oral routes. The ADCs or ADC derivatives can be administered, for example by infusion or bolus injection, by absorption through epithelial or mucocutaneous linings (e.g., oral mucosa, rectal and intestinal mucosa, and the like) and can be administered together with other biologically active agents such as chemotherapeutic agents. Administration can be systemic or local.

In specific embodiments, the anti-LGALS3BP ADC or ADC derivative composition is administered by injection, by means of a catheter, by means of a suppository, or by means of an implant, the implant being of a porous, non-porous, or gelatinous material, including a membrane, such as a silastic membrane, or a fiber. Typically, when administering the composition, materials to which the ADC or ADC derivative does not absorb are used.

In other embodiments, the ADC or ADC derivative is delivered in a controlled release system. In one embodiment, a pump may be used (101, 102, 103, 104). In another embodiment, polymeric materials can be used. (105, 106, 107, 108, 109, 110). Other controlled release systems are discussed, for example, in Langer, supra.

The anti-LGALS3BP ADCs or ADC derivatives are administered as pharmaceutical compositions comprising a therapeutically effective amount of the ADC or ADC derivative and one or more pharmaceutically compatible ingredients. For example, the pharmaceutical composition typically includes one or more pharmaceutical carriers (e.g., sterile liquids, such as water and oils, including those of petroleum, animal, vegetable or synthetic origin, such as peanut oil, soybean oil, mineral oil, sesame oil and the like). Water is a more typical carrier when the pharmaceutical composition is administered intravenously. Saline solutions and aqueous dextrose and glycerol solutions can also be employed as liquid carriers, particularly for injectable solutions. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in ref. 111. Such compositions will contain a therapeutically effective amount of the nucleic acid or protein, typically in purified form, together with a suitable amount of carrier so as to provide the form for proper administration to the patient. The formulations correspond to the mode of administration.

In typical embodiments, the pharmaceutical composition is formulated in accordance with routine procedures as a pharmaceutical composition adapted for intravenous administration to human beings. Typically, compositions for intravenous administration are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration.

In certain embodiments, the pharmaceutical compositions comprising the anti-LGALS3BP ADC or ADC derivative can further comprise a second therapeutic agent (e.g., a second ADC or ADC derivative or a non-conjugated cytotoxic or immunosuppressive agent such as, for example, any of those described herein).

The amount of the ADC or ADC derivative that is effective in the treatment of LGALS3BP-expressing cancer can be determined by standard clinical techniques. In addition, in vitro assays may optionally be employed to help identify optimal dosage ranges. The precise dose to be employed in the formulation will also depend on the route of administration, and the stage of LGALS3BP-expressing cancer, and should be decided according to the judgment of the practitioner and each patient's circumstances. Effective doses may be extrapolated from dose-response curves derived from in vitro or animal model test systems.

For example, toxicity and therapeutic efficacy of the ADCs or ADC derivatives can be determined in cell cultures or experimental animals by standard pharmaceutical procedures for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. ADCs or ADC derivatives that exhibit large therapeutic indices are preferred. Where an ADC or ADC derivative exhibits toxic side effects, a delivery system that targets the ADC or ADC derivative to the site of affected tissue can be used to minimize potential damage non-LGALS3BP-expressing cells and, thereby, reduce side effects.

The data obtained from animal studies can be used in formulating a range of dosage for use in humans. The dosage of the anti-LGALS3BP ADC or ADC derivative typically lies within a range of circulating concentrations that include the ED₅₀ with little or no toxicity. The dosage may vary within this range depending upon the dosage form employed and the route of administration utilized. For any ADC or ADC derivative used in the method, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Generally, the dosage of an anti-LGALS3BP ADC or ADC derivative administered to a patient with a LGALS3BP-expressing cancer is typically 0.1 mg/kg to 100 mg/kg of the subject's body weight. More typically, the dosage administered to a subject is 0.1 mg/kg to 50 mg/kg of the subject's body weight, even more typically 1 mg/kg to 30 mg/kg, 1 mg/kg to 20 mg/kg, 1 mg/kg to 15 mg/kg, or 1 mg/kg to 10 mg/kg of the subject's body weight. Generally, human or humanized antibodies have a longer half-life within the human body than antibodies from other species due to the immune response to the foreign proteins. Thus, lower dosages of ADCs comprising humanized, chimeric or human antibodies and less frequent administration is often possible.

The anti-LGALS3BP ADC or ADC derivative can be administered in combination with one or more other therapeutic agents for the treatment or LGALS3BP-expressing cancers. For example, combination therapy can include a second cytostatic, cytotoxic, or immunosuppressive agent (for example, an unconjugated cytostatic, cytotoxic, or immunosuppressive agent such as those conventionally used for the treatment of cancers or immunological disorders). Combination therapy can also include, e.g., administration of an agent that targets a receptor or receptor complex other than LGALS3BP on the surface of LGALS3BP-expressing cancer cells. An example of such an agent is a second, non-LGALS3BP antibody that binds to a molecule at the surface of LGALS3BP-expressing cancer cells. Another example is a ligand that targets such a receptor or receptor complex. Typically, such an antibody or ligand binds to a cell surface receptor on LGALS3BP-expressing cancer cells and enhances the cytotoxic effect of the anti-LGALS3BP antibody by delivering a cytotoxic signal to LGALS3BP-expressing cancer cells.

Such combinatorial administration can have an additive or synergistic effect on disease parameters (e.g., severity of a symptom, the number of symptoms, or frequency of relapse).

With respect to therapeutic regimens for combinatorial administration, in a specific embodiment, an anti-LGALS3BP ADC or ADC derivative is administered concurrently with a second therapeutic agent. In another specific embodiment, the second therapeutic agent is administered prior or subsequent to administration of the anti-LGALS3BP ADC or ADC derivative, by at least an hour and up to several months, for example at least an hour, five hours, 12 hours, a day, a week, a month, or three months, prior or subsequent to administration of the ADC or ADC derivative.

The 1959-sss-DM3 ADC of the present invention and the pharmaceutical composition containing this 1959-sss-DM3 ADC have an excellent safety profile. For example, when the ADC was administered to 5 mg/Hg to cross-breed rabbits as a single i.v. injection, no toxicity findings were observed as a result of observation until day 7 after the administration.

The present invention is not to be limited in scope by the specific embodiments described herein. Various modifications of the invention in addition to those described herein will become apparent to those skilled in the art from the foregoing description and accompanying figures. Such modifications are intended to fall within the scope of the appended claims.

The invention is further described in the following examples, which are in not intended to limit the scope of the invention. Cell lines described in the following examples were maintained in culture according to the conditions specified by the American Type Culture Collection (ATCC) or Deutsche Sammlung von Mikroorganismen and Zellkulturen GmbH, Braunschweig, Germany (DMSZ). Cell culture reagents were obtained from Invitrogen Corp., Carlsbad, Calif.

### EXAMPLES

### Starting Materials.

1. Antibody 1959-sss (C220S-C226S-C229S) at 11 mg/ml in PBS, pH 7.4 The Antibody1959-sss was used at the concentration of 11 mg/ml in PBS pH 7.4.
2. The maytansinoids DM1, DM3 and DM4, as SH-derivatives were purchased from XDCEXPLORER CO., LTD (Shanghai, China).

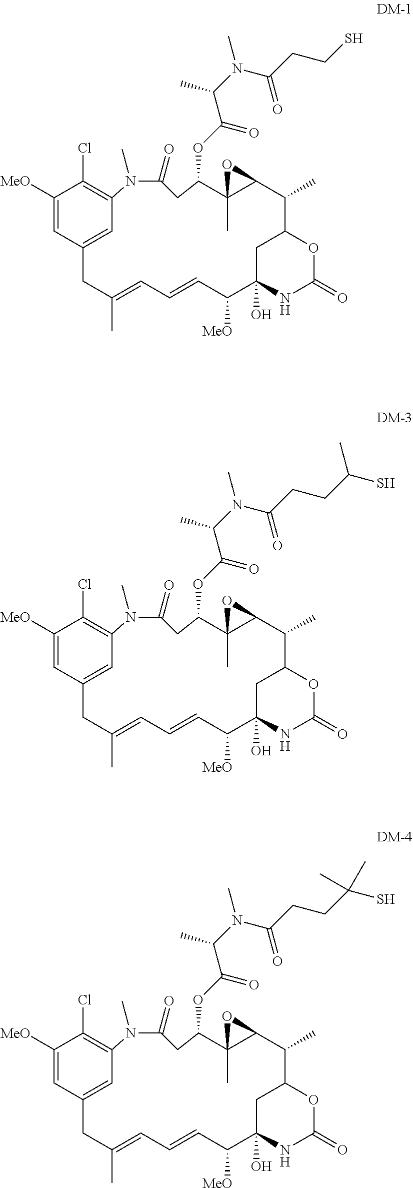 [SMILES: COc1cc2cc(c1Cl)N(C)C(=O)C[C@H](OC(=O)[C@H](C)N(C)C(=O)CCS)[C@]1(C)O[C@H]1[C@H](C)C1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)CCC(C)S)[C@]1(C)O[C@H]1[C@H](C)C1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)CCC(C)(C)S)[C@]1(C)O[C@H]1[C@H](C)C1C[C@@](O)(NC(=O)O1)[C@H](OC)/C=C/C=C(\C)C2]

Following is the description of the conjugation procedure of these maytansinoids to 1959-sss antibody, using DM3 as a cytotoxic drug. The procedure is identical when using DM1 or DM4.

### Example 1. Reduction of the of the Antibody 1959-Sss Followed by DNTB Derivatization

### Materials and Methods:

Antibody 1959-sss was reduced using 60 molar excess of TCEP (tris(2-carboxyethyl) phosphine (Sigma-Aldrich), stock dissolved in phosphate-buffered saline, pH=7.4 (PBS). The reaction was carried out overnight at room temperature (about 25° C.).

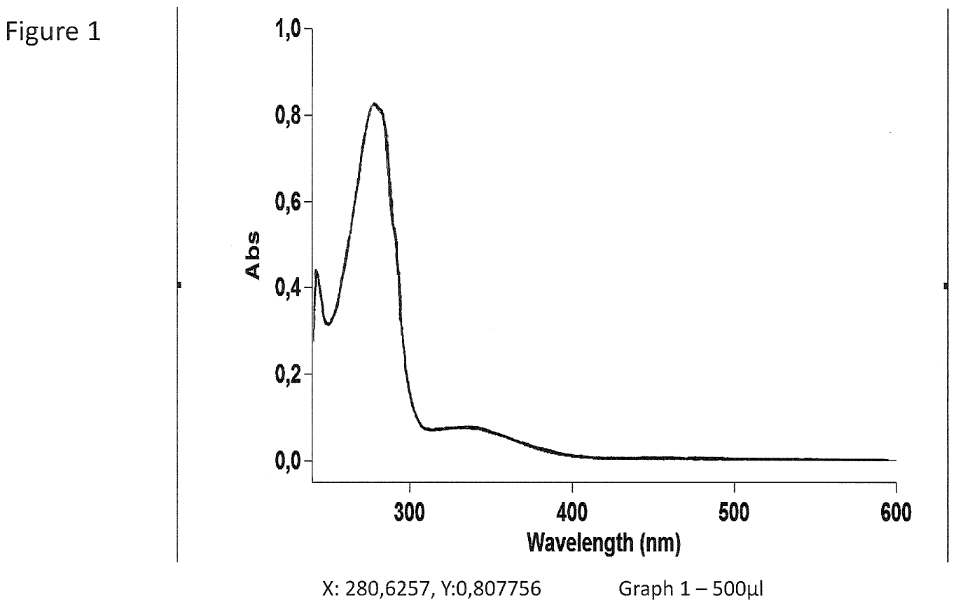

For DTNB derivatization, the TCEP-reduced antibody was first supplemented with 1M phosphate buffer pH 7.4 up to 100 mM final phosphate. DTNB (5,5′-Dithiobis(2-nitrobenzoic acid) (Sigma-Aldrich) stock (26.7 mg/ml in EtOH) was then added to obtain 100 molar excess over the antibody. The reaction was carried out overnight at room temperature. The reaction was stopped by passing the 1959-sss/DTNB mixture through a G25 Sephadex column equilibrated in PBS/5% sucrose/10% DMA (NN′ dimethyl acetamide, SIGMA) to eliminate unreacted DTNB. The protein concentration was analysed by UV-VIS spectrum to evaluate the peak at 280 nm indicating the presence of the antibody (FIG. 1).

### Results:

1959-sss was reacted with 100 molar excess DTNB and the reaction stopped by passage through a G25 column. The spectrum represents the protein after the G25 column, showing that the derivatization reaction does not destabilize the protein.

### Example 2. Separation of the Complex 1959-Sss-DM3 from Unreacted Free DM3

### Materials and Methods

DM3 conjugation. The 1959-sss DTNB derivatized antibody was reacted with 10 molar excess DM3 (stock 1 mg/ml in DMA, Sigma-aldrich) in PBS/5% sucrose/10% DMA overnight at room temperature. The reaction was stopped adding to the mixture 500 molar excess iodoacetamide (Sigma-aldrich).

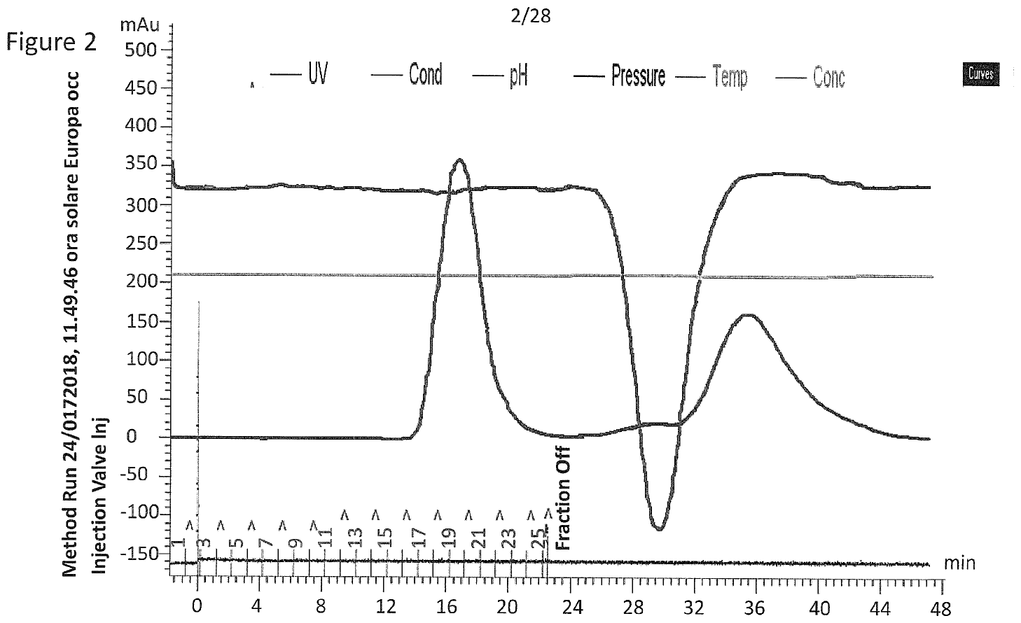

For the purification of the 1959-sss-DM3, the reaction mixture was passed through a G25 Sephadex column equilibrated in PBS/5% sucrose/10% DMA in an isocratic way with a flow rate of 1 ml/min, collecting 1 ml fractions (FIG. 2).

After incubation with 10 molar excess DM3, the complex 1959-sss-DM3 was separated from unreacted free DM3 by gel filtration. The first peak in the chromatogram (minutes 15-20) contained 1959-sss-DM3, while the second peak (minutes 30-40) contained unreacted free DM3.

### Results

The complex 1959-sss-DM3 was separated from unreacted free DM3 by gel filtration. The first peak in the chromatogram (minutes 15-20) contained 1959-sss-DM3, while the second peak (minutes 30-40) contained unreacted free DM3.

### Example 3. Absorbance Spectrum of the Antibody 1959-Sss after the Reaction with DM3

### Materials and Methods

1959-sss was reacted with 10 molar excess DM3 and the reaction stopped by passage through a G25 column.

### Results

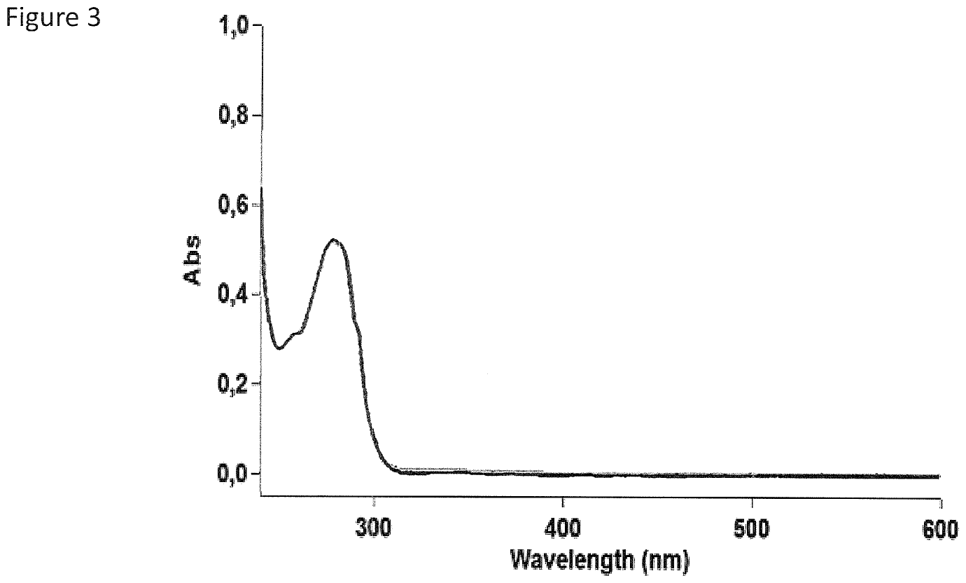

The spectrum represents the protein after the G25 column and shows that also this second derivatization reaction does not destabilize the protein (FIG. 3).

### Example 4. Calibration Curve Construction by HPLC

### Materials and Methods

Based on HPLC-C18 (Vertex plus column, Knauer) analysis at 254 nm relative to DM3 release after the reduction with 60 molar excess TCEP, we estimated a DAR of 2 DM3 molecules per antibody molecule. The protocol used was:

### TFA 0.1% (Sigma-Aldrich)

### TFA 0.1%+Acetonitrile 80% (Sigma-Aldrich)

Detected at 254 (0.80 ml/min) using a gradient as follow (table 1):

TABLE 1

| Step | A % | B % | Time (min) |
|---|---|---|---|
| I | 100 | 0 | (LOAD) |
| 1 | 100 | 0 | 0-10 |
| 2 | 0 | 100 | 10-40 |
| 3 | 0 | 100 | 40-45 |
| 4 | 100 | 0 | 45-50 |
| 5 | 100 | 0 | 50-60 |

### Results

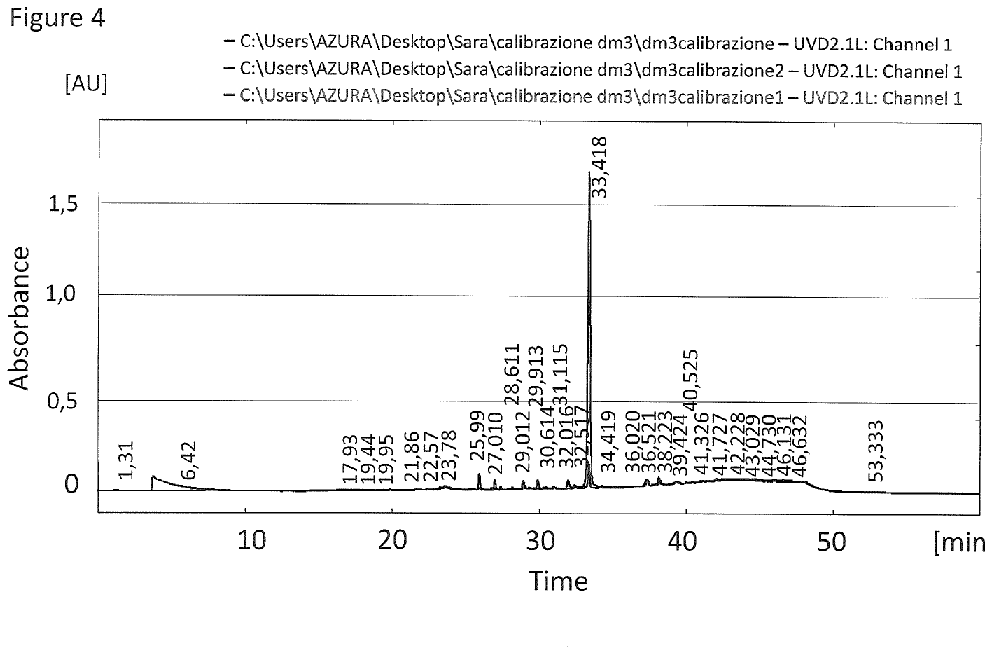

500 ml of solutions containing different amounts of free DM3 were analyzed by HPLC chromatography on a C18 column. The area of the peak at minute 33 was used to determine a calibration curve that was used to estimate DM3 released from the conjugate 1959-sss-DM3 (FIG. 4).

### Example 5. HPLC Analysis of DM3 Release from 1959-Sss-DM3 Conjugate Following Reduction with TCEP and Calculation of DAR (Drug Antibody Ratio)

### Materials and Methods

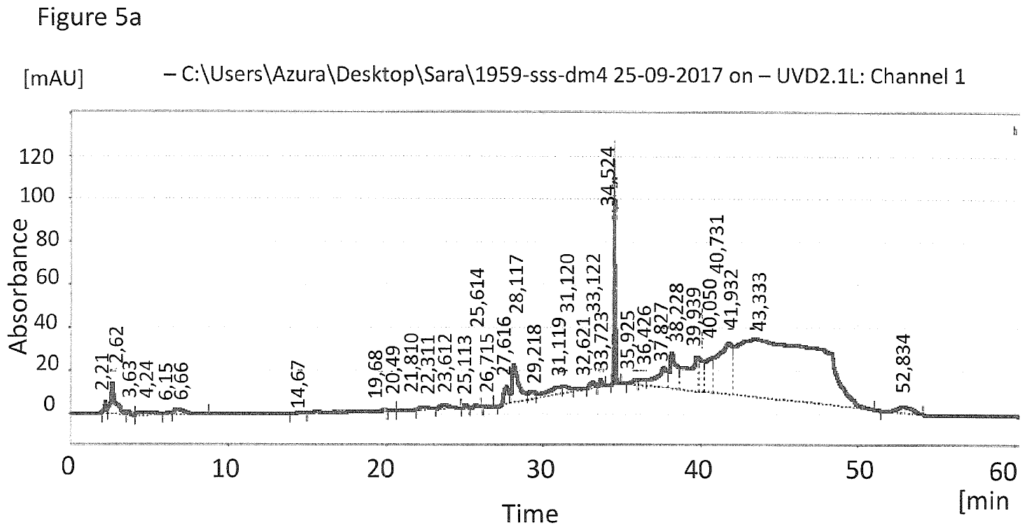

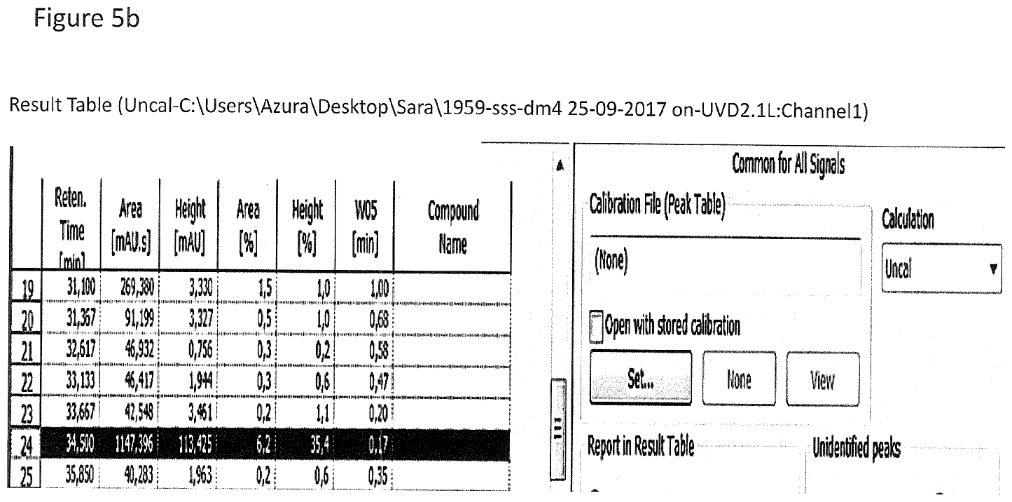

500 ml (0.4 mg/ml) of 1959-sss-DM3 not reduced and reduced were analyzed on a C18 HPLC column and the peak corresponding to DM3 (minute 33) (FIG. 5) released from reduced 1959-sss-DM3 interpolated within the calibration curve.

### Results

The Ab1959-SSS-DM3 DAR was calculated to be 2.

### Example 6. HIC-HPLC Analysis of Unconjugated 1959-Sss and 1959-Sss-DM3

### Materials and Methods

Naked 1959-sss antibody and the conjugated 1959-sss-DM3 were analyzed by HIC chromatography at the following conditions:

A: 1.5 M Ammonium sulphate, 50 mM sodium phosphate pH 7.0, 5% isopropanol
B: 50 mM sodium phosphate pH7, 20% isopropanol. Gradient as shown in Table 2.

TABLE 2

| Step | A % | B % | Time (min) |
|---|---|---|---|
| I | 100 | 0 | (LOAD) |
| 1 | 0 | 100 | 0-20 |
| 2 | 100 | 0 | 20.1-35 |

### Results

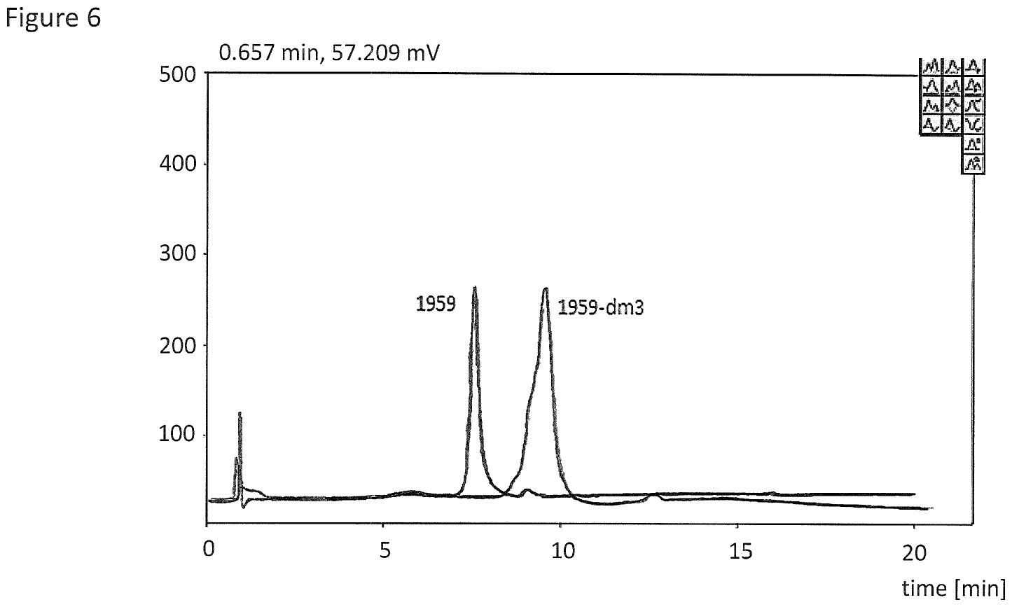

The chromatogram shows the presence of single species in both samples (FIG. 6), demonstrating that the conjugation reaction gave rise to a homogeneous product (green peak) completely avoid of free (unconjugated) 1959-sss antibody.

### Example 7. MALDI Mass Spectrometry Analysis

### Materials and Methods

Antibody 1959-sss and ADC 1959-sss-DM3 were desalted by PD Spin TrapG25 and a few microliters were used for the MALDI mass analysis. Briefly, 2 microliters of each sample were mixed with 2 microliters of a s-DHB saturated solution in 0.1% TFA in distilled water/acetonitrile (50:50). Mixtures were deposited on a stainless steel target and let dry. Mass spectra were acquired using an Ultraflex MALDI TOF/TOF (Bruker, GmBH) in linear positive mode.

### Results

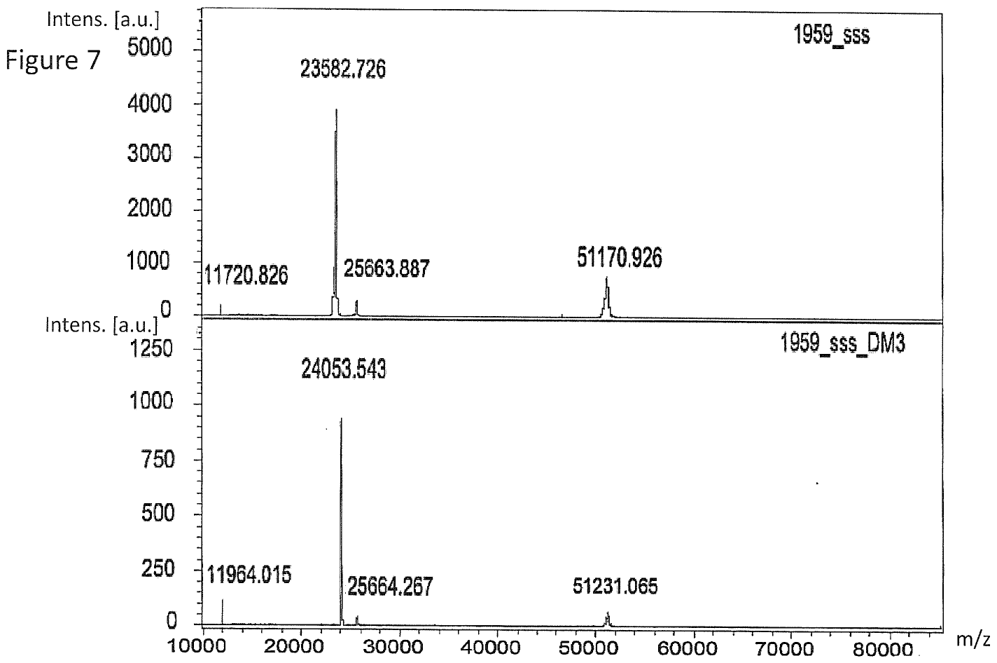

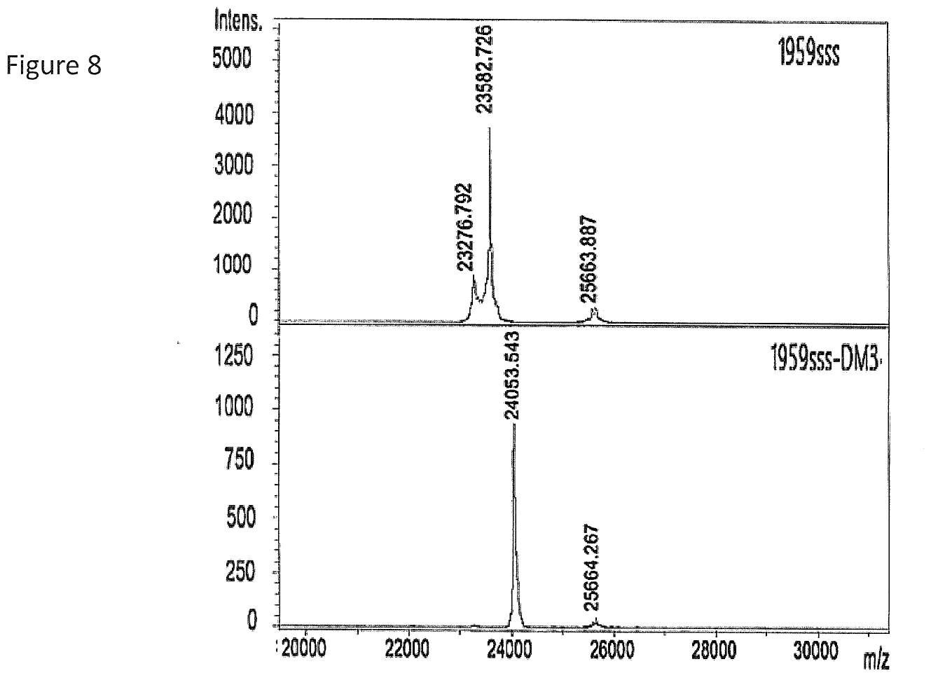

MALDI mass spectrometry was done on naked and conjugated forms of 1959-sss antibody. As expected (FIG. 7), the heavy and light chains were easily separated in the absence of any reduction, being the interchain disulphides absent in the engineered antibody. The heavy chains appeared as a peak of mass 51200 Da in both samples. Light chains instead showed heterogeneity in the naked 1959-sss antibody (see also FIG. 8), while were represented by a single peak in the conjugated 1959-sss-DM3 antibody.

### Example 8. Light Chains MALDI Mass Spectrometry Analysis of Naked 1959-sss and Conjugated 1959-sss-DM3

### Materials and Methods (See Example 7)

### Results

As shown in FIG. 8, light chains from naked 1959-sss are represented by two peaks with masses of 23276 and 23582, while light chains from 1959-sss-DM3 are represented by a single peak of mass 24053 Da.

### Example 9. MALDI Mass Spectrometry Analysis of 1959-Sss after Reduction with TCEP

### Materials and Methods (See Example 7)

### Results

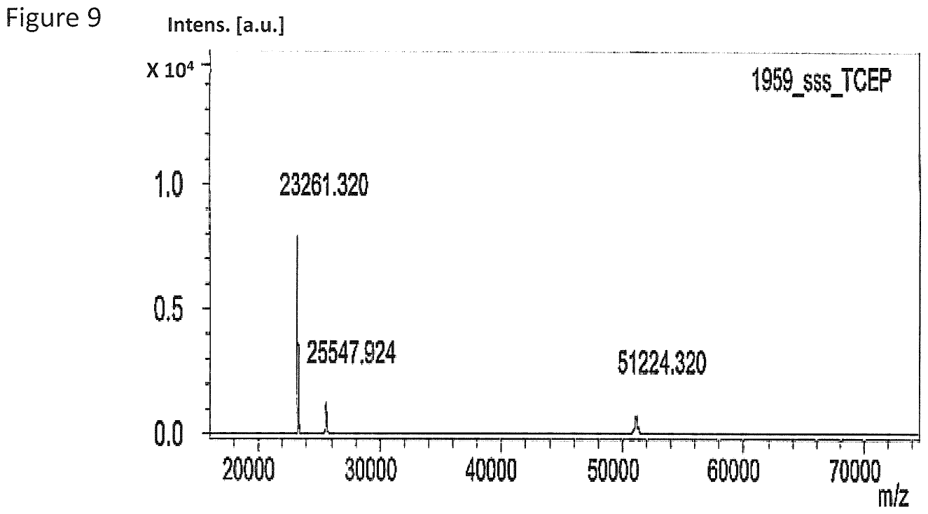

This profile shows that upon reduction with TCEP, the two peaks observed for 1959-sss naked antibody homogeneously shift to a single peak of mass 23261 Da (FIG. 9). The difference of about 320 Da between light chains may be due to the release of glutathione, possibly remaining from the expression of recombinant 1959-sss in CHO cells.

### Example 10. HIC Chromatography of 1959-Sss-DM3, 1959-Sss-DM3 after Reduction with TCEP, Naked 1959-Sss

### Materials and Methods (See Example 6)

### Results

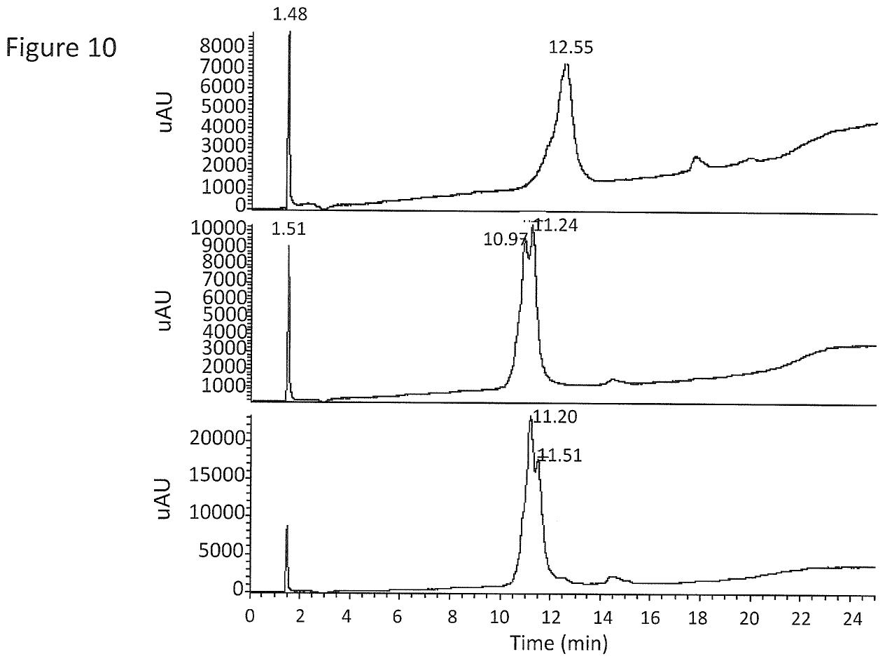

FIG. 10 shows antibody 1959-sss-DM3 before (upper panel) and after reduction (middle panel) in comparison with naked 1959-sss (lower panel). TCEP reduction of 1959-sss-DM3 shifts the position of the peak down to that corresponding to the naked antibody.

### Example 11. Binding of Unconjugated 1959-Sss and 1959-Sss-DM3 to LGALS3BP

### Materials and Methods

Human recombinant LGALS3BP (2 μg/ml) was pre-coated overnight at 4° C. on 96 well-plates NUNC Maxisorp modules. After blocking with 1% BSA in PBS containing 0.1% Tween-20 for 1 hour at room temperature, unconjugated 1959-sss antibody or 1959-sss-DM3 was added and incubated at the indicated concentration for 2 hours at room temperature. After several washes with PBS-0.1% Tween-20, anti-human IgG-HRP was added and incubated for 1 hour at room temperature. After washes, stabilized chromogen was added for at least 10 minutes in the dark, before stopping the reaction with the addition of 1 N H₂SO₄. The resulting color was read at 492 nm with an Elisa reader.

### Results

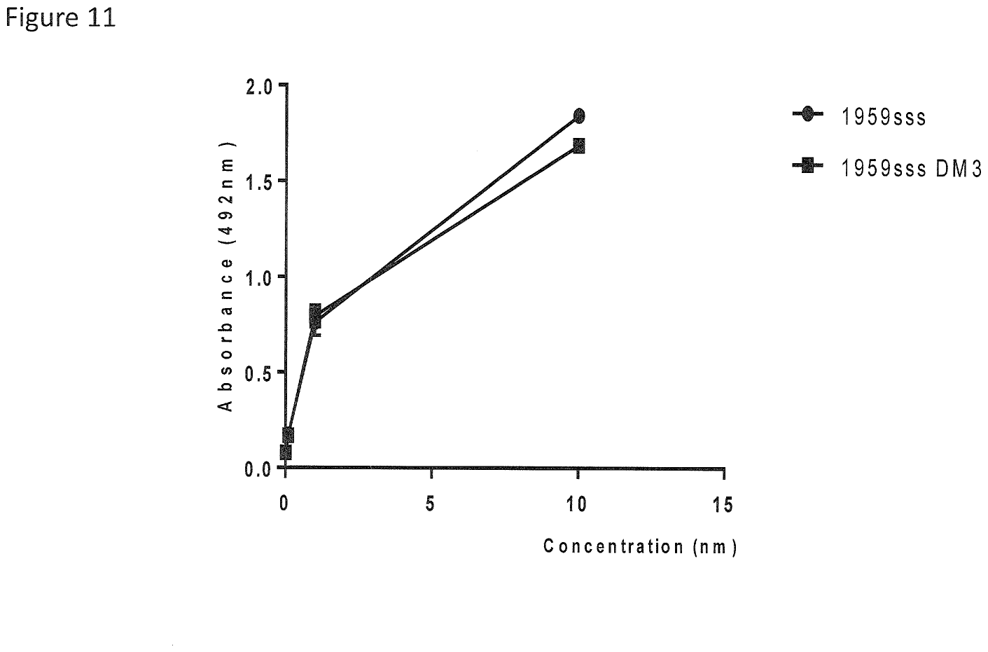

Unconjugated 1959-sss and 1959-sss DM3 displays the same binding behaviour to LGALS3BP (FIG. 11).

### Example 12. Expression of LGALS3BP in Human Tumor Cell Lines

### Materials and Methods

Human tumor cells were grown on glass coverslips for 24 hours. Coverslips were incubated for 2 hours at room temperature with 1959-sss or 1959-sss-DM3 3. At the end of incubation, cells were fixed in 4% paraformaldehyde for 15 minutes at room temperature, permeabilized with 0.25% Triton X-100 for 5 minutes, and blocked with 0.1% BSA for 1 hour at room temperature. Coverslips were then incubated with anti-human IgG-Alexa Fluor 488 conjugated. DRAQS was used to visualize nuclei. Images were acquired with a Zeiss LSM 510 meta-confocal microscopy using 488- and 633-nm lasers.

A, C, E, G, unconjugated 1959-sss; B, D, F, H, 1959-sss-DM3.
A-B; MDA-MB-231 breast cancer; C-D, A375 melanoma; E-F, FADU, H&N cancer;
G-H, HBF, normal bronchial fibroblasts.

### Results:

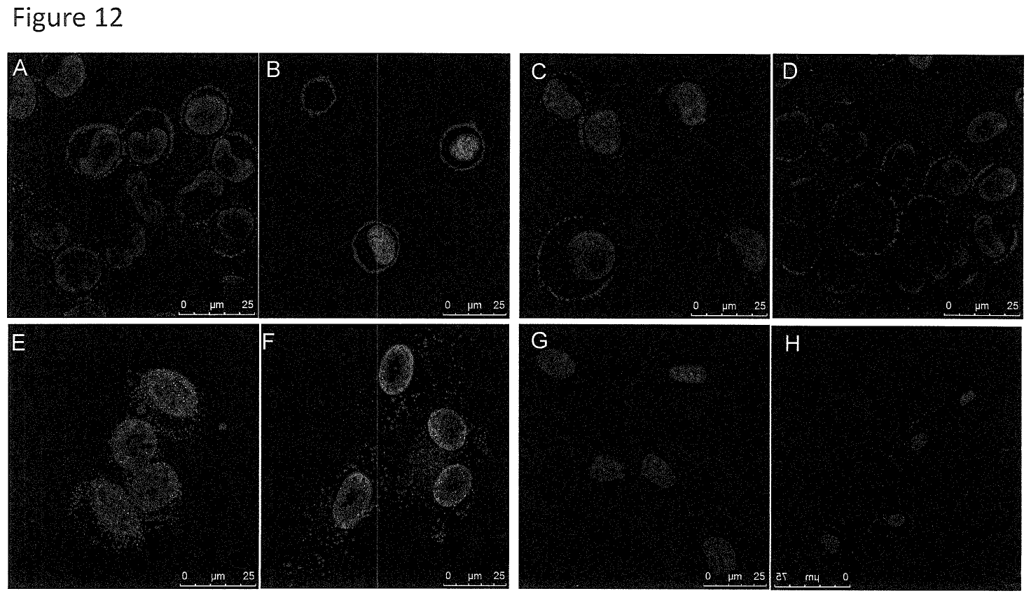

FIG. 12 shows staining at the cell membrane of human tumor cells, but not normal cells upon incubation with 1959-sss-ADC or unconjugated antibody followed by anti-human fluorescently labelled IgG. Staining was granular, possibly indicating clustering induced by LGALS3BP upon secretion.

### Example 13: Co-Localization of LGALS3BP with CD63 and CD81 in Human Melanoma Cells

### Materials and Methods:

A375 human tumor cells were grown on glass coverslips for 24 hours. Coverslips were washed with PBS, and cells fixed with 4% paraformaldehyde for 15 minutes at room temperature. After washing twice with PBS, cells were incubated in 3% bovine serum albumin in PBS at room temperature for 20 min. Then, cells were incubated with the following antibodies at 4° C. overnight: (A), anti-CD63 (from mouse, Thermo Fisher, diluted 1:50), (B), anti-human CD81 (from mouse, Thermo Fisher, diluted 1:20), anti-human Gal-3BP (1959-sss, diluted at 2 □g/ml). After washing with PBS, coverslips were then incubated with either Alexa Fluor 633 anti-human IgG (Invitrogen) or Alexa Fluor 488 anti-mouse IgG (Invitrogen) at room temperature for 30 min. DAPI was used to visualize nuclei. Images were acquired with a TCS SP5 Leica-confocal microscopy.

### Results:

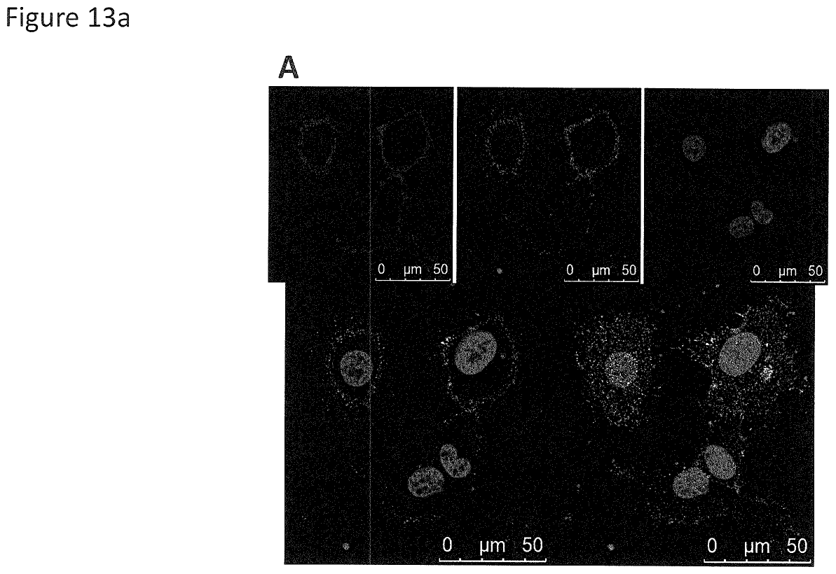

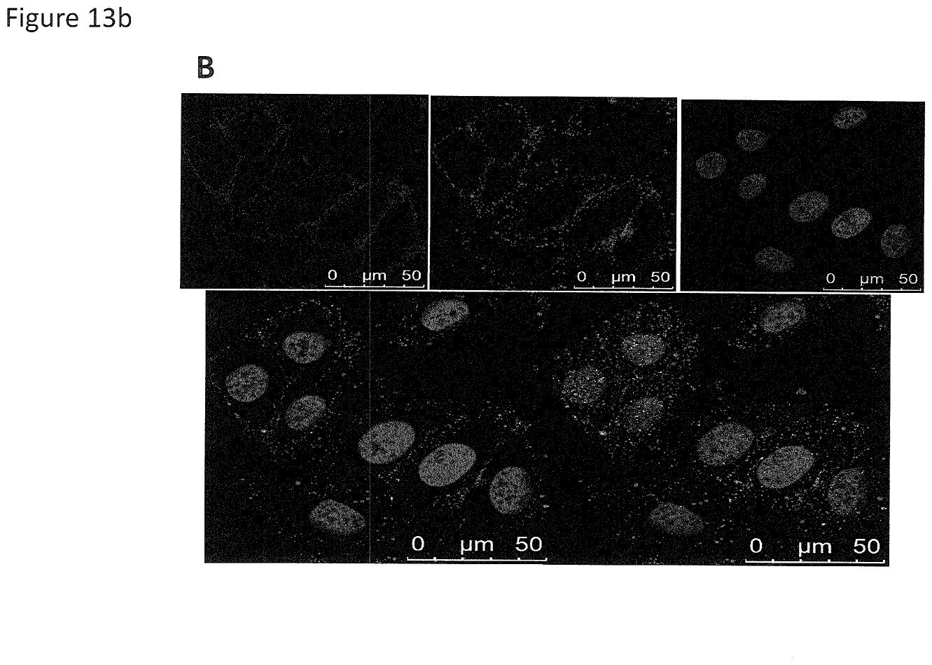

FIG. 13 shows staining at the cell membrane of human melanoma cells upon incubation with 1959-sss alone or in combination with CD 63 and CD81 followed by either anti-human fluorescently labelled IgG or anti-mouse fluorescently labelled IgG. LGALS3BP co-localizes with the exosome marker proteins at the cell membrane. Staining was granular, possibly indicating clustering induced by LGALS3BP upon secretion.

### Example 14. In Vivo Therapeutic Efficacy of 1959-Sss-DM1 and 1959-Sss-DM3 in a Xenog Raft Model of Melanoma

### Materials and Methods

Human melanoma xenografts were established by injecting subcutaneously 5×10⁶ A375 cells in 5-6 week old CD-1 nu/nu mice nude mice. When the average tumor size within a group was approximately 100 mm³, mice were divided into four groups in a manner to provide a similar range of tumor size in each group. The groups were treated with PBS (control), unconjugated 1959-sss antibody (10 mg/kg), 1959-sss-DM1 (10 mg/kg), or 1959-sss-DM3 (10 mg/kg). Treatments were administered by intravenous injection daily for 5 days (arrows). Tumor volumes were calculated according to the following formula: tumor volume=(length*width²)/2. Tumor volumes were monitored every week. A, tumor growth curves. The data represent mean tumor volume (±SEM), n=5 or 6 mice/group. B, body weight of mice groups shown in panel A.

### Results

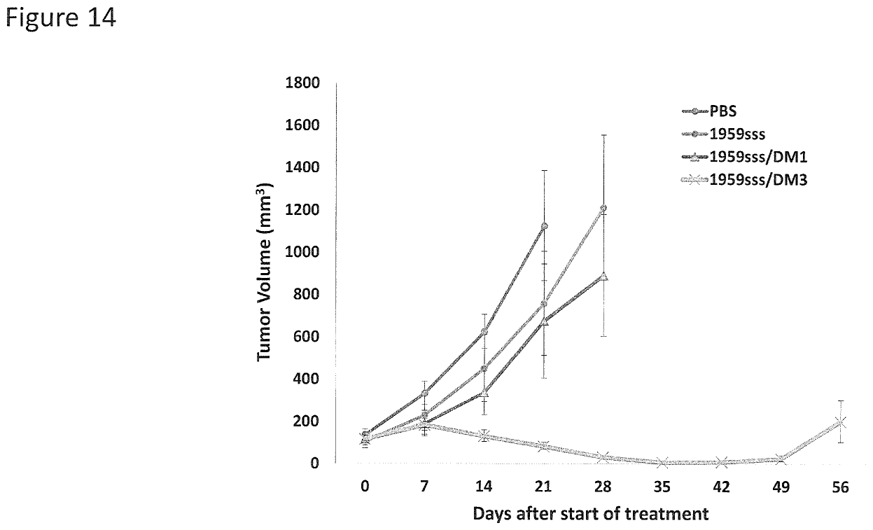

FIG. 14 shows no therapeutic activity in mice treated with unconjugated 1959-sss antibody. Very little therapeutic activity was detected with 1959-sss-DM1, as tumor growth rate in this group was not significantly different to either control group or unconjugated 1959-sss antibody group. Treatment of mice with 1959-sss-DM3 significantly inhibited tumor growth. Difference between 1959-sss DM3 and control, P<0.00001.

### Example 15. In Vivo Therapeutic Efficacy of 1959-Sss-DM3 and 1959-Sss-DM4 in a Xenog Raft Model of Melanoma

### Materials and Methods

A, growth of subcutaneously implanted human A375 melanoma xenografts in nude mice treated with PBS (control), 1959-sss-DM3 (10 mg/kg) daily or twice a week (t/w) for a total of 5 injections, or 1959-sss-DM4 (10 mg/Kg) t/w for a total of 5 injections. The data represent mean tumor volume (±SEM), n=5 or 6 mice/group. B, Kaplan-Meyer plot showing survival of the treatment groups shown in panel A.

### Results

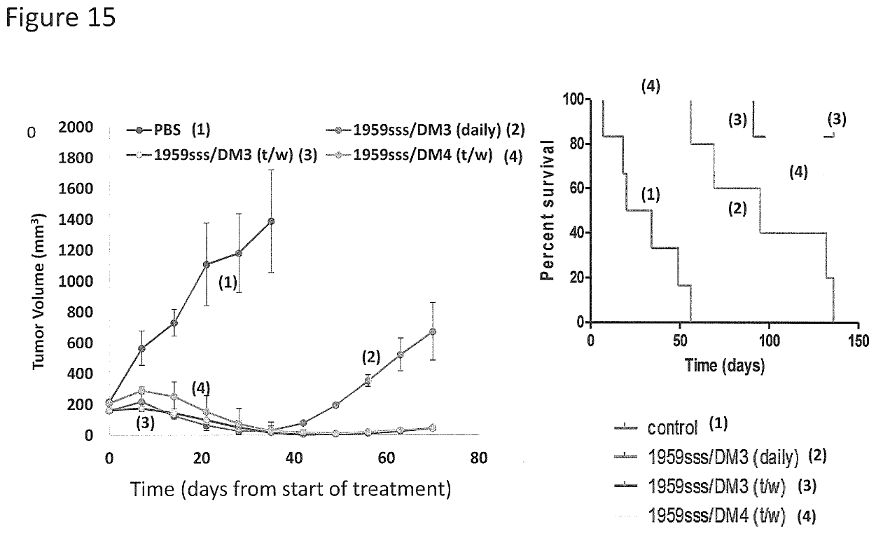

FIG. 15 shows that treatment of mice with 1959-sss-DM3, or 1959-sss-DM4 induced a significant inhibition of tumor growth. Difference between 1959-sss DM3 or 1959-sss-DM4.

Mice treated with 1959-sss based ADC survived longer than control mice. Difference between 1959-sss DM3 or1959-sss-DM4 t/w vs. control, P<0.00001; 1959-sss-DM3 daily vs. control, P<0.0001.

At 140 days from start of treatment, the number of mice displaying complete remission (CR), i.e. no palpable tumor is indicated in Table 3.

TABLE 3

| | Cut off | CR |
|---|---|---|
| | (TV about 1.5 cm³) | (not palpable tumor) |
| PBS | 6/6 | |
| 1959sss DM3 (daily) | 5/6 | 0 |
| 1959sss DM3 (t/w) | 1/6 | 5/6 |
| 1959sss DM4 (t/w) | 3/6 | 3/6 |

### Example 16. Dose-Response of 1959-Sss-DM3 in a Xenograft Model of Melanoma

### Materials and Methods

Growth of subcutaneously implanted human A375 melanoma xenografts in nude mice treated with PBS (control), or 1959-sss-DM3 at 10, 3 or 1 mg/Kg t/w for a total of 5 injections.

### Results

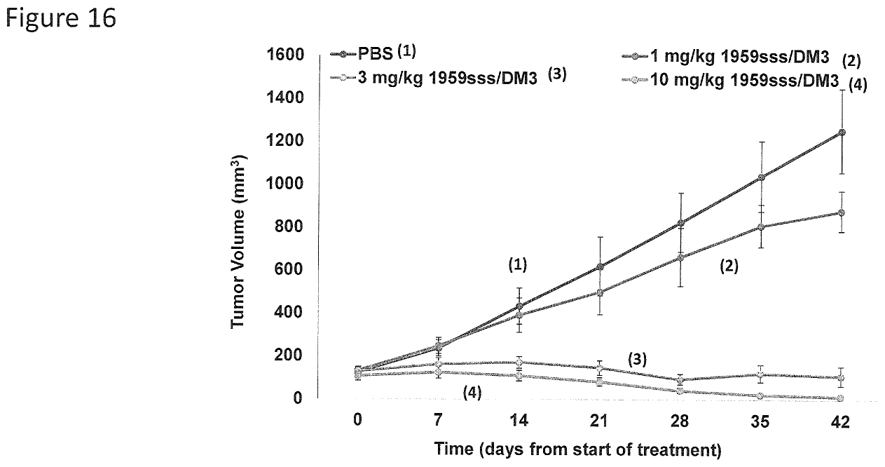

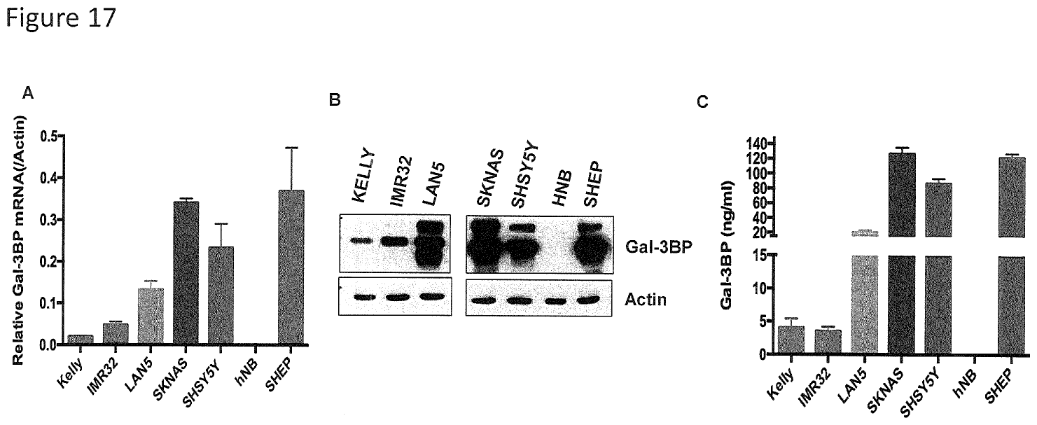

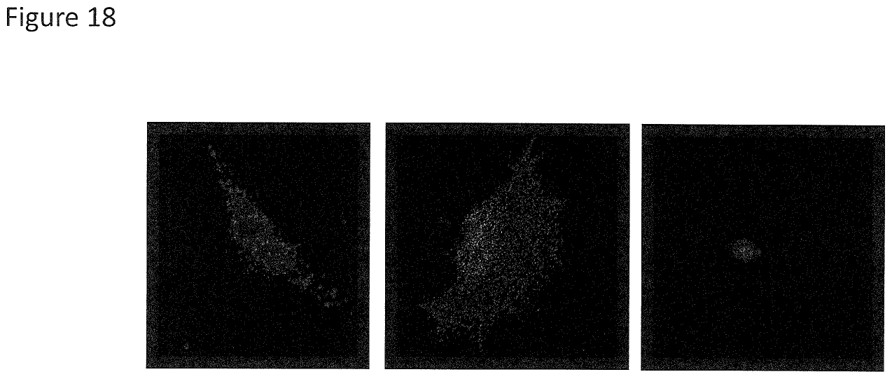

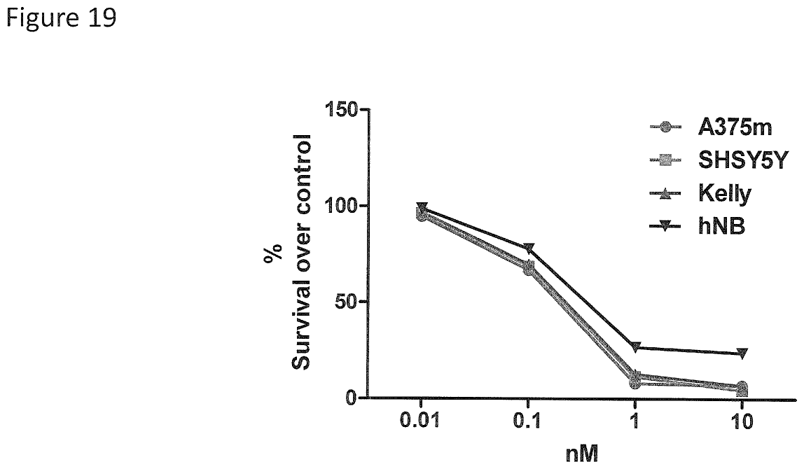

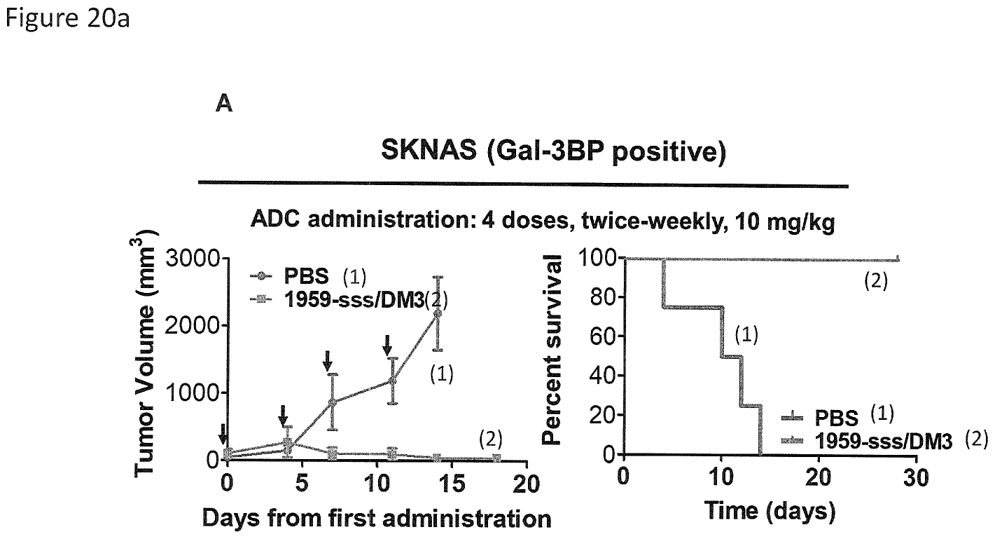

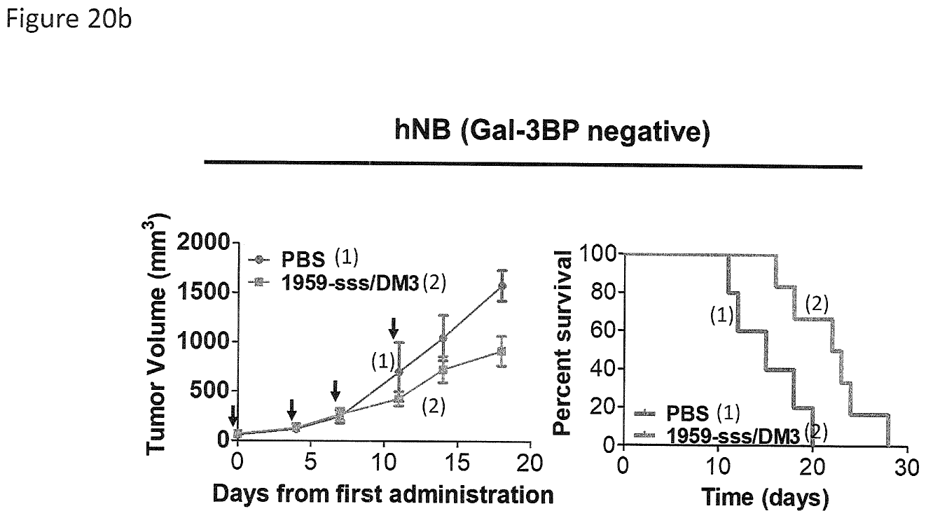

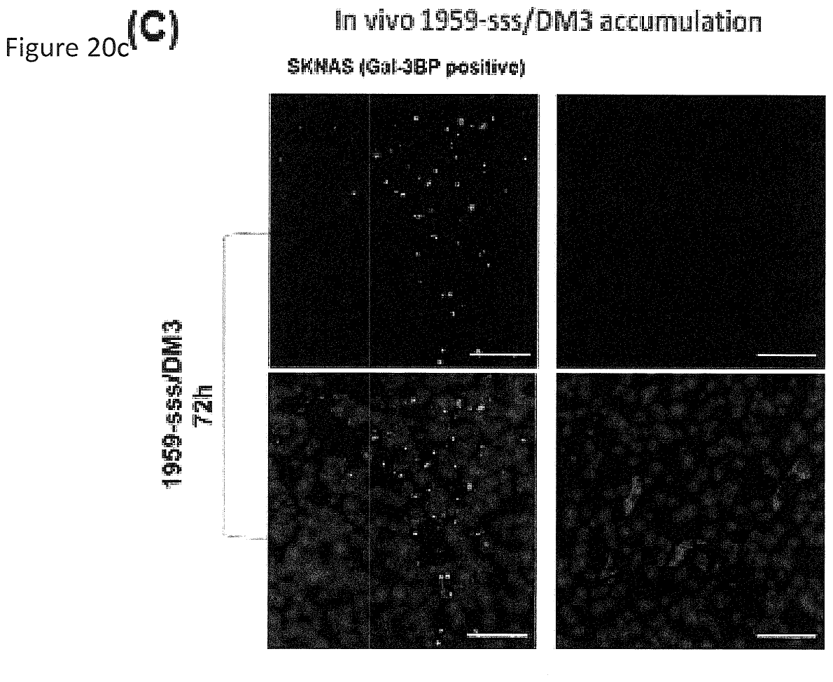

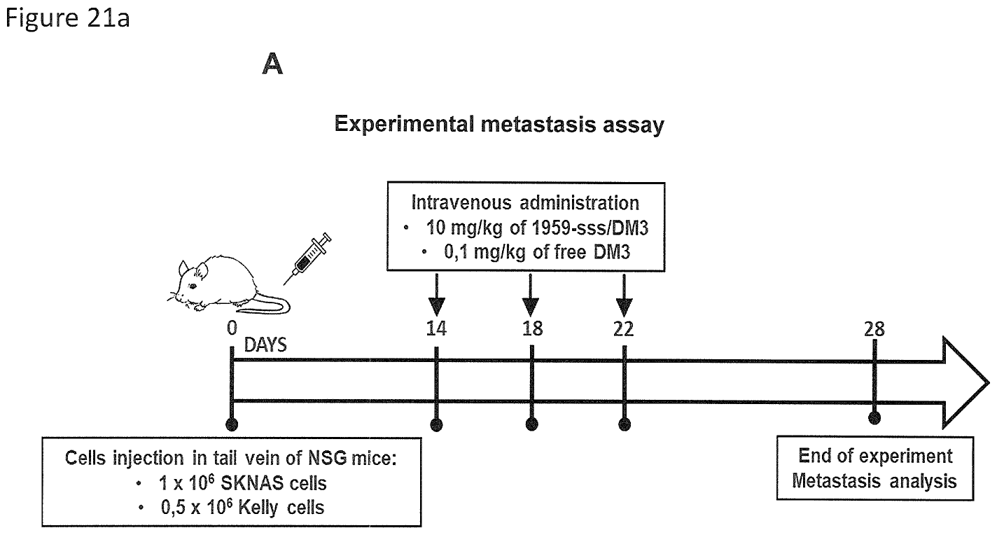

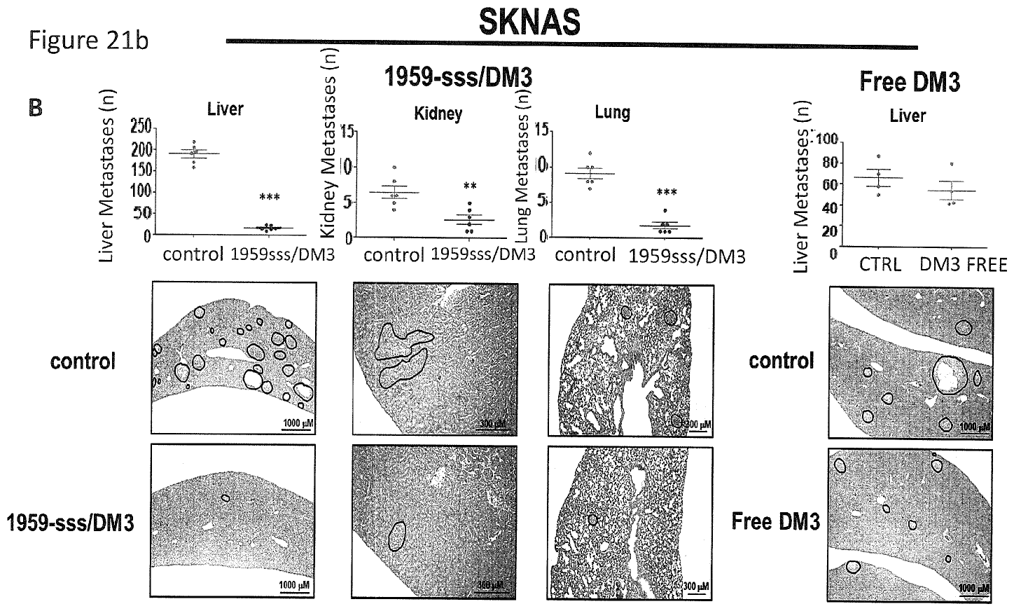

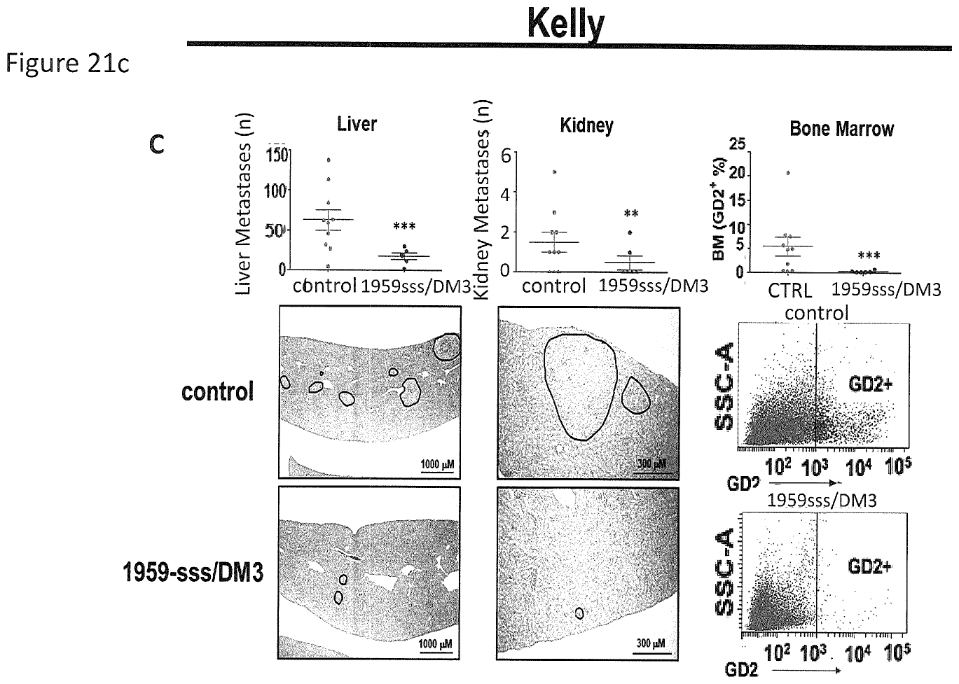

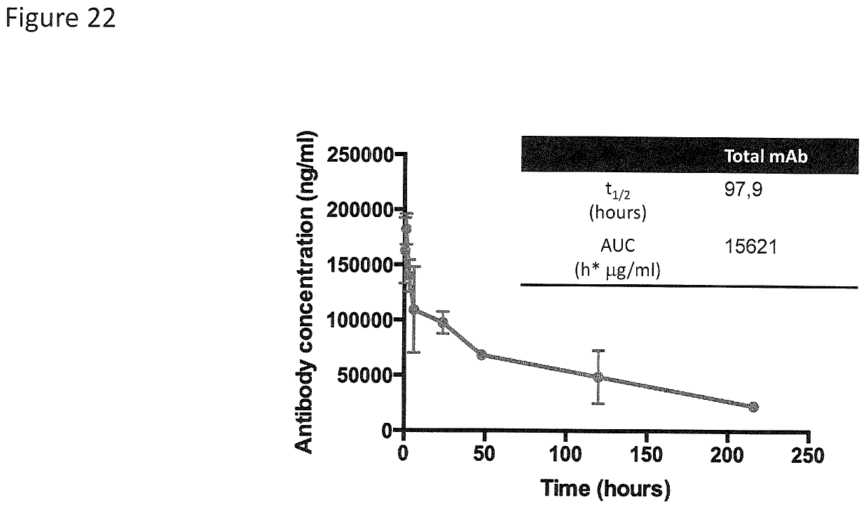

Treatment of mice with 1959-sss DM3 at 3 mg/Kg or 10 mg/Kg induced a similar tumor growth inhibition vs. control (P<0.0001) (FIG. 16).

### Example 17: LGALS3BP (Gal-3BP) is Expressed and Secreted by Neuroblastoma Cells

### Materials and Methods:

Neuroblastoma cell lines were cultured in complete culture medium; after 48 hours, cell pellets and supernatants were collected. For Real-Time PCR (A), total RNA was extracted by cells with RNeasy Mini Kit, and 1 μg of RNA was reverse transcribed by HyperScript™ Reverse Transcriptase according to manufacturer's instructions. The RNA quality and quantity were assessed by NanoDrop spectrometer. Real-time PCR was performed with SsoAdvanced Universal SYBR® Green Supermix using the following primers: LGALS3BP Fw 5′-gaacccaaggcgtgaacgat-3′ (SEQ ID NO:12), Rw 5′-gtcccacaggttgtcacaca-3′ (SEQ ID NO:13). LGALS3BP mRNA expression was calculated relative to human β-actin as housekeeping gene; the primers used were Act Fw 5′-cagctcaccatggatgatgatatc-3′ (SEQ ID NO:14) and Rw 5′-aagccggccttgcacat-3′ (SEQ ID NO:15) with an amplification protocol as follows: one cycle of 95° C. for 30 sec and 40 cycles of 95° C. for 15 s and 60° C. for 30 sec on Real-Time Detection System CFX96. Relative mRNA expression, normalized to an endogenous reference β-actin, was determined using the −Δct method.

For Western Blot (B) pellets were lysed with RIPA lysis buffer supplemented with protease and phosphatase inhibitors for 10 min at 4° C. Insoluble materials were removed by centrifugation (13,000 rpm for 10 min at 4° C.) and protein concentration assessed by the method of Bradford. Equal amounts of total protein were subjected to SDS-PAGE and then transferred to nitrocellulose membranes. The membranes were blocked with 5% non-fat dry milk in PBS with 0.1% Tween 20 and incubated overnight using anti-β-Actin and anti-LGALS3BP antibodies. Circulating LGALS3BP in supernatant cells was measured by sandwich ELISA (C) provided by Diesse Diagnostica Senese Spa (Siena, Italy), following the manufacturer's instructions.

All cell lines were obtained by Amerycan Type Culture Collection (ATCC), except primary HB cell lines described by Chaiwatanasikul et al (Cell Death Dis. 2011 Oct. 20; 2:e219. doi: 10.1038/cddis.2011.99).

### Results:

Six out of seven neuroblastoma cell lines examined express and secrete LGALS3BP, as evaluated by (A) RNA and (B) WB or by ELISA (C) as secreted protein in the cuture medium. Expression and secretion differ among the neuroblastoma cell lines, except in primary human NB cell line which resulted to be negative for Gal-3BP.

### Example 18: 1959 Antibody Specifically Stains LGALS3BP at the Membrane of LGALS3BP Positive but not Negative Neuroblastoma Cells

### Materials and Methods:

Neuroblastoma cell lines (Kelly, SKNAS and hNB) were plated in coverslips and grown in complete culture medium for 24 h. Thereafter, cells were incubated with 10 μg/ml of 1959-sss antibody at 37° C. for 90 min. At the end of the incubation, cells were fixed in 4% paraformaldehyde, and then stained with anti-human AlexaFluor 488 conjugated secondary antibody. Draq5 was used to visualize nuclei.

### Results:

1959 antibody stains LGALS3BP at the membrane of positive but not negative LGALS3BP cells.

### Example 19: Maytansine-Derivative SH-DM3 is Active in Neuroblastoma Cells

### Materials and Methods:

Neuroblastoma cell lines (SHSYSY, Kelly and hNB) and A375m melanoma cell line were plated and treated with increasing concentrations of SH-DM3 for 72 hours. The cell killing activity of the drug was evaluated by MTT assay.

### Results:

SH DM3 displays potent cell killing activity in vitro on neuroblastoma cell lines respect to A375m melanoma cells, used as a positive control.

### Example 20: 1959-Sss/DM3: Therapeutic Activity in Neuroblastoma is Target Dependent

### Materials and Methods

A) SKNAS cells (LGALS3BP-positive) and hNB cells (LGALS3BP-negative) derived xenograft models were generated by subcutaneous injection into the right flank of mice of 3×10⁶ of cells in 200 μl of PBS. When xenografts became palpable, animals were divided into 2 groups in a way to provide a similar range of tumor size for each group. The treated group received 4 intravenous injections twice weekly of 1959-sss/DM3 (10 mg/kg) whereas the control group received PBS only. Tumor volume was monitored every week by a caliper and calculated by the following formula: tumor volume (mm³)=(length×width²)/2. A tumor volume of 2 cm³ was chosen as endpoint for both experiments after which mice were sacrificed. Survival curves were derived from Kaplan-Meier estimates and compared by log-rank test (GraphPad Prism 5). B) 1959-sss/DM3 accumulation in tumour tissues was evaluated by immunofluorescence analysis of SKNAS and hNB tumour xenografts. Animals bearing tumours received a single injection of 1959-sss/DM3 at the dose of 10 mg/kg and thereafter animals were sacrificed 72 h later. Fresh tumour tissues were frozen in a cryo-embedding medium and cryostat sections were stained with anti CD31/CD105 antibodies (red) to visualize blood vessels; cells nuclei were stained by DRAQS (blue). Scale bars: 50 μm.

### Results:

Administration of 1959sss/DM3 at the dose of 10 mg/kg induces shrinkage in LGALS3BP-positive but not negative neuroblastoma cell derived tumor xenografts. In line with this, after 72 hours from an intravenous injection of 1959sss/DM3, immunofluorescence of LGALS3BP-positive, but not LGALS3BP-negative tumor tissues shows accumulation of the antibody-drug conjugate. These data suggest that LGALS3BP expression may be a primary determinant of tumor shrinkage in response to 1959-sss/DM3 treatment in vivo.

### Example 21: 1959-Sss/DM3: Therapeutic Activity in Experimental Metastasis Neuroblastoma Models

### Materials and Methods:

Schematic representation of experimental metastasis assays (A) performed in SKNAS (B) and Kelly (C) neuroblastoma cell models. Eight-weeks old NSG mice were injected in the tail vein with neuroblastoma cells, the intravenous treatments (1959-sss/DM3 or free DM3 at the indicated doses, PBS as control) were started after 14 days from the injection for 4 injections twice weekly. After 28 days, mice were sacrificed, and organs (liver, kidney, lung and bone marrow) were processed for metastasis analysis. Liver, kidney and lung were harvested, fixed in 10% neutral buffered formalin, paraffin embedded, sectioned and stained with Hematoxylin and Eosin. All neuroblastoma metastatic lesions were analyzed and plotted on graphs (above). Representative images are shown (below). As regards bone marrow analysis, femora and tibiae were cut out from mice after sacrifice, soft tissue were accurately removed and bone marrow cells suspension was collected into a tube by flushing the shaft with 1 ml of cold PBS using a 1 ml syringe with needle. Bone marrow cells were resuspended and washed several times, then cells were stained with an anti-human-GD2 followed by fluorescent secondary antibody for flow cytometry analysis. Percentage of GD2 positive cells is shown in the dot plot.

### Results:

Treatment with 1959-sss/DM3 at the dose of 10 mg/kg potently inhibits formation of neuroblastoma metastatic lesions, while treatment with a dose equivalent to the free drug load on ADC at 10 mg/kg had no significant impact on the reduction of the metastatic lesions, confirming that there is a therapeutic advantage in the use of 1959-sss/DM3.

### Example 22: 1959-Sss/DM3 Pharmacokinetic

### Materials and Methods:

Non-tumour bearing Athymic CD-1 nu/nu mice were injected intravenously with a single dose of 1959-sss/DM3 (10 mg/kg) and blood samples collected at different time points thereafter. Serum concentrations of total antibody were measured by sandwich ELISA using as capture antigen recombinant LGALS3BP and goat anti-human IgG-HRP for detection. Half-time (t_{1/2}) and AUC values were obtained by Kinetica 5.0 software.

### Results:

Pharmacokinetic studies in mouse serum reveals that 1959sss/DM3 ADC possesses a Half-time of around 97.9 hours.

### REFERENCES

1. lacobelli S, Bucci I, D'Egidio M, Giuliani C, Natoli C, Tinari N, Rubistein M, Schlessinger J. Purification and characterization of a 90 kDa protein released from human tumors and tumor cell lines. FEBS Lett. 1993 Mar. 15; 319(1-2):59-65.
2. Grassadonia A, Tinari N, lurisci I, Piccolo E, Cumashi A, Innominato P, D'Egidio M, Natoli C, Piantelli M, lacobelli S. 90K (Mac-2 BP) and galectins in tumor progression and metastasis.
3. Piccolo E, Tinari N, Semeraro D, Traini S, Fichera I, Cumashi A, La Sorda R, Spinella F, Bagnato A, Lattanzio R, D'Egidio M, Di Risio A, Stampolidis P, Piantelli M, Natoli C, Ullrich A, lacobelli S. LGALS3BP, lectin galactoside-binding soluble 3 binding protein, induces vascular endothelial growth factor in human breast cancer cells and promotes angiogenesis. J Mol Med (Berl). 2013 January; 91(1):83-94.
4. Rabinovich G A, Baum L G, Tinari N, Paganelli R, Natoli C, Liu F T, lacobelli S. Galectins and their ligands: amplifiers, silencers or tuners of the inflammatory response? Trends Immunol. 2002 June; 23(6):313-20.
5. Stampolidis P, Ullrich A, lacobelli S.LGALS3BP, lectin galactoside-binding soluble 3 binding protein, promotes oncogenic cellular events impeded by antibody intervention. Oncogene. 2015 Jan. 2; 34(1):39-52.
6. Lee J H, Bae J A, Lee J H, Seo Y W, Kho D H, Sun E G, Lee S E, Cho S H, Joo Y E, Ahn K Y, Chung I J, Kim K K. Glycoprotein 90K, downregulated in advanced colorectal cancer tissues, interacts with CD9/CD82 and suppresses the Wnt/beta-catenin signal via ISGylation of beta-catenin. Gut. 2010 July; 59(7):907-17.
7. Sasaki T, Brakebusch C, Engel J, Timpl R. Mac-2 binding protein is a cell-adhesive protein of the extracellular matrix which self-assembles into ring-like structures and binds beta1 integrins, collagens and fibronectin. EMBO J. 1998 Mar. 16; 17(6):1606-13.
8. Becker r et al. Tumor stroma marker endosialin (Tem1) is a binding partner of metastasis-related protein Mac-2 BP/90K. FASEB J. 2008 August; 22(8):3059-67.
9. Laubli H, Alisson-Silva F, Stanczak M A, Siddiqui S S, Deng L, Verhagen A, Varki N, Varki A. Lectin galactoside-binding soluble 3 binding protein (LGALS3BP) is a tumor-associated immunomodulatory ligand for CD33-related Siglecs. J Biol Chem. 2014 Nov. 28; 289(48):33481-91.
10. Piccolo E, Tinari N, D'Addario D, Rossi C, lacobelli V, La Sorda R, Lattanzio R, D'Egidio M, Di Risio A, Piantelli M, Natali P G, lacobelli S. Prognostic relevance of LGALS3BP in human colorectal carcinoma. J Transl Med. 2015 Jul. 30; 13:248.
11. Inohara H¹, Akahani S, Koths K, Raz A. Interactions between galectin-3 and Mac-2-binding protein mediate cell-cell adhesion. Cancer Res. 1996 Oct. 1; 56(19):4530-4.
12. Traini S, Piccolo E, Tinari N, Rossi C, La Sorda R, Spinella F, Bagnato A, Lattanzio R, D'Egidio M, Di Risio A, Tomao F, Grassadonia A, Piantelli M, Natoli C, lacobelli S. Inhibition of tumor growth and angiogenesis by SP-2, an anti-lectin, galactoside-binding soluble 3 binding protein (LGALS3BP) antibody. Mol Cancer Ther. 2014 April; 13(4):916-25.
13. WO2012052230A1
14. Sala G, Rapposelli I G, Ghasemi R, Piccolo E, Traini S, Capone E, Rossi C, Pelliccia A, Di Risio A, D'Egidio M, Tinari N, Muraro R, lacobelli S. EV20, a Novel Anti-ErbB-3 Humanized Antibody, Promotes ErbB-3 Down-Regulation and Inhibits Tumor Growth In Vivo. Transl Oncol. 2013 Dec. 1; 6(6):676-84
15. WO 93/17715
16. WO 92/08802
17. WO 91/00360
18. WO 92/05793
19. Tutt, A., Stevenson, G., and Glennie, M. (1991) Trispecific F(ab′)₃ derivatives that use cooperative signaling via the TCR/CD3 complex and CD2 to activate and redirect resting cytotoxic T cells. J. Immunol. 147, 60-69
20. Kostelny, S. A., M. S. Cole, and J. Y. Tso. 1992. Formation of a bispecific antibody by the use of leucine zippers. J. Immunol. 148: 1547-1553.29.
21. WO 92/22324
22. R. L. Mullinax, E. A. Gross, B. N. Hay, J. R. Amberg, M. M. Kubitz, J. A. Sorge. Expression of a heterodiraeric Fab antibody protein in one single cloning step. BioTechniques, 12(1992), pp. 864-869.
23. Better M, Chang C P, Robinson R R, Horwitz A H. Escherichia Coli secretion of an active chimeric antibody fragment. Science. 1988 May 20; 240(4855):1041-3.
24. Sawai et al, AJRI, vol. 34,1995, pages 26-34
25. U.S. Pat. No. 4,946,778A
26. U.S. Pat. No. 5,258,498A
27. Huston J S, Mudgett-Hunter M, Tai M S, McCartney J, Warren F, Haber E, Oppermann H. Methods Enzymol. 1991; 203:46-88. Protein engineering of single-chain Fv analogs and fusion proteins.
28. Shu L, Qi C F, Schlom J, et al. Secretion of a single-gene-encoded immunoglobulin from myeloma cells. Proc Natl Acad Sci USA. 1993; 90:7995-7999. 28.
29. Skerra, A., and A. Plückthun. 1988. Assembly of a functional immunoglobulin Fv fragment in Escherichia coli. Science 240:1038-1040
30. L. Morrison, Transfectomas provide novel chimeric antibodies, Science 229: 1202 (1985).
31. Oi V T, Morrison S L. Chimeric antibodies. Biotechniques 1986; 4:214. 228
32. Gillies S D, Lo K M, Wesolowski J. High-level expression of chimeric antibodies using adapted cDNA variable region cassettes. J Immunol Methods 1989; 125: 191-202
33. U.S. Pat. No. 5,807,715A
34. U.S. Pat. No. 4,816,567A
35. U.S. Pat. No. 4,816,397A
36. U.S. Pat. No. 5,585,089A
37. Riechmann L, Clark M, Waldmann H, Winter G. Nature. 1988 Mar. 24; 332(6162):323-7. Reshaping human antibodies for therapy.
38. EP 0 239 400
39. WO 91/09967
40. U.S. Pat. No. 5,225,539A
41. U.S. Pat. No. 5,530,101A
42. U.S. Pat. No. 5,585,089A
43. EP 0 592 106
44. EP 0 519 596
45. Padlan E A. A possible procedure for reducing the immunogenicity of antibody . . . Mol Immunol 1991; 28(4-5):489-498
46. Studnicka et al. Human-engineered monoclonal antibodies retain full specific binding activity by preserving non-CDR complementarity-modulating residues. ‘PROTEIN ENGINEERING, vol. 7, no. 6, June 1994 OXFORD, GB, pages 805-814,
47. Roguska et al., Humanization of murine monoclonal antibodies through variable domain resurfacing” 1994, PNAS 91:969-973.
48. U.S. Pat. No. 5,565,332A
49. U.S. Pat. No. 4,444,887A
50. U.S. Pat. No. 4,716,111A
51. WO 98/46645
52. WO 98/50433
53. WO 98/24893
54. WO 98/16654
55. WO 96/34096
56. WO 96/33735
57. WO 91/10741
58. Jespers L S, et al., Guiding the selection of human antibodies from phage display repertoires to a single epitope of an antigen. Biotechnology 1994; 12:899-903
59. Lonberg N, Huszar D. Int Rev Immunol. 1995; 13(1):65-93. Human antibodies from transgenic mice.;
60. WO 92/01047;
61. EP 0 598,877;
62. U.S. Pat. No. 5,413,923A;
63. U.S. Pat. No. 5,625,126A;
64. U.S. Pat. No. 5,633,425A;
65. U.S. Pat. No. 5,569,825A
66. U.S. Pat. No. 5,661,016A;
67. U.S. Pat. No. 5,545,806A;
68. U.S. Pat. No. 5,814,318A;
69. U.S. Pat. No. 5,885,793A;
70. U.S. Pat. No. 5,916,771A;
71. U.S. Pat. No. 5,939,598A
72. U.S. Pat. No. 5,155,027A;
73. U.S. Pat. No. 5,336,603A;
74. U.S. Pat. No. 5,359,046A
75. U.S. Pat. No. 5,349,053A;
76. EP 0 367 166;
77. WO 96/04388
78. U.S. Pat. No. 4,676,980A
79. Ausubel et al., eds., Short Protocols in Molecular Biology (John Wiley & Sons, Inc., New York, 4th ed. 1999)
80. Harlow & Lane, Using Antibodies: A Laboratory Manual (Cold Spring Harbor Laboratory Press, Cold Spring Harbor, N.Y., 1999.)
81. WO 86/05807;
82. WO 89/01036;
83. U.S. Pat. No. 5,122,464A
84. Foecking M K, Hofstetter H. Powerful and versatile enhancer-promoter unit for mammalian expression vectors. Gene 1986; 45:101-105. 52
85. Cockett M I, Bebbington C R, Yarranton G T. 1990. High level expression of tissue inhibitor of metalloproteinases in Chinese hamster ovary cells using glutamine synthetase gene amplification. Bio/Technology 8: 662-667.
86. Current Protocols in Molecular Biology (Ausubel et al. eds., John Wiley & Sons, N.Y., 1993)
87. Gene Transfer and Expression, A Laboratory Manual (Stockton Press, N.Y., 1990);
88. Current Protocols in Human Genetics (Dracopoli et al. eds., John Wiley & Sons, N.Y., 1994, Chapters 12 and 13)
89. Colberre-Garapin, F., F. Horodniceanu, P. Kourilsky, and A. C. Garapin. 1981. A new dominant hybrid selective marker for higher eukaryotic cells. J. Mol. Biol. 150:1-14. 9.
90. Bebbington & Hentschel, The Use of Vectors Based on Gene Amplification for the Expression of Cloned Genes in Mammalian Cells in DNA Cloning, Vol. 3 (Academic Press, New York, 1987).
91. Crouse, G. F., R. N. McEwan, and M. L. Pearson. 1983. Expression and amplification of engineered mouse dihydrofolate reductase minigenes. Mol. Cell. Biol. 3:257-266.
92. Proudfoot, N., Transcriptional Interference and Termination Between Duplicated a-Globin Gene Constructs Suggest a Novel Mechanism for Gene Regulation, Nature 322:52, 1986.
93. Kohler G., Immunoglobulin Chain Loss in Hybridoma Lines, PNAS 77:2197, 1980.
94. Wilson, I. A, Niman, H L Houghten, R A, et al., 1984, The structure of an antigenic determinant in a protein, Cell 37:767-778
95. Monoclonal Antibodies For Immunotargeting Of Drugs In Cancer Therapy,” in Monoclonal Antibodies And Cancer Therapy (Reisfeld et al. eds., Alan R. Liss, Inc., 1985)
96. Hellstrom et al., “Antibodies For Drug Delivery,” in Controlled Drug Delivery (Robinson et al. eds., Marcel Dekker, Inc., 2nd ed. 1987)
97. Thorpe, “Antibody Carriers Of Cytotoxic Agents In Cancer Therapy: A Review,” in Monoclonal Antibodies '84: Biological And Clinical Applications (Pinchera et al. eds., 1985)
98. “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985)
99. Thorpe P. E. & Ross W. C. J. (1982) The preparation and cytotoxic properties of antibody-toxin conjugates. Immunol. Rev. 62, 119-58
100. WO 89/12624
101. Langer, R., 1990, New methods of drug delivery, Science 249:1527-1533. Lewis, D. H., 1990
102. Sefton M V Crit Rev Biomed Eng. 1987; 14(3):201-40. Implantable pumps.
103. Buchwald, H., Rohde, T. D., Warco, R. L., et al.: Long-term continuous intravenous heparin administration by an implantable infusion pump in ambulatory patients with recurrent venous thrombosis. Surgery. 88:507-516, 1980
104. Saudek, C. D/, J. L, Selam, H. A, Pitt, et al. 1989. A preliminary trial of the programmable implantable medication system for insulin delivery. N. Engl. J. Med. 321: 574-579
105. Medical Applications of Controlled Release (Langer & Wise eds., CRC Press, Boca Raton, Fla., 1974)
106. Controlled Drug Bioavailability, Drug Product Design and Performance (Smolen & Ball eds., Wiley, New York, 1984)
107. Ranger & Peppas, 1983, Macromol. Sci. Rev. Macromol. Chem. 23:61
108. Levy R J, Wolfrum J, Schoen F J, et al: Inhibition of calcification of bioprosthetic heart valves by local controlled release diphosphonate. Science 228:190-192, 1985
109. During J, Freese A, Sabel B, Saltzman W, Deutch A, Roth R, Langer R, Controlled release of dopamine from a polymeric brain implant: In vivo characterization. 1989, Ann. Neurol. 25:351
110. Howard, M. A. et al. 1989. Intracerebral drug delivery in rats with lesion-induced memory deficits. J. Neurosurg 71:105
111. Remington's pharmaceutical sciences. XIII ed. Editor-in-Chief Eric W. Martin

1. A targeted therapeutic agent of the formula:
B-D
or a pharmaceutically acceptable salt thereof, wherein
B is a non-internalizing binding moiety specific for a cancer associated protein; and
D is a cytotoxic drug moiety.

2. The targeted therapeutic agent according to claim 1, wherein the binding moiety B is not an antibody or an antibody fragment.

3. The targeted therapeutic agent according to claim 1, wherein the cancer associated protein is secreted LGALS3BP.

4. The targeted therapeutic agent according to claim 1, wherein the binding moiety B is a multivalent binding moiety having two or more ligands for binding to a target entity.

5. The targeted therapeutic agent according to claim 1, wherein the binding moiety B comprises a non-internalizing antibody.

6. The targeted therapeutic agent according to claim 5, wherein the non-internalizing antibody is specific for LGALS3BP.

7. The targeted therapeutic agent according to claim 1, wherein the cytotoxic drug moiety D is selected from a tubulin disruptor.

8. The therapeutic agent according to claim 1, wherein the non-internalizing binding moiety B is conjugated to the cytotoxic drug moiety D via a linker.

9. The targeted therapeutic agent according to claim 8, wherein the linker comprises a cysteine group for linking to the drug moiety through a disulfide bond.

10. The targeted therapeutic agent according to claim 1, wherein the cytotoxic drug moiety D in active form comprises a thiol group for forming a disulfide bond to the binding moiety B or linker in the compound.

11. The targeted therapeutic agent according to claim 1, wherein said agent when administered to balb/c nu/nu mice having subcutaneous A375 tumors daily for five consecutive days or twice a week after a total of 5 injections causes a significant tumor shrinkage lasting for at least 50 days.

12. The targeted therapeutic agent according to claim 1, wherein the agent when administered to balb/c nu/nu mice having subcutaneous SKNAS tumors twice a week after a total of 4 injections causes significant tumor shrinkage.

13. The targeted therapeutic agent according to claim 1, wherein the agent when administered to NSG immunodeficient mice having metastases deriving from intravenous injection of SKNAS cells or Kelly cells twice a week after a total of 3 injections causes a significant reduction of the number and size of metastatic deposits.

14. The targeted therapeutic agent according to claim 1, for use in the treatment of a neoplastic disease.

15. The targeted therapeutic agent for use according to claim 14, wherein the neoplastic disease is a LGALS3BP-expressing tumor.

16. A pharmaceutical composition comprising a targeted therapeutic agent according to claim 1 and a pharmaceutically acceptable carrier.

17. The targeted therapeutic agent of claim 5, wherein the non-internalizing antibody is a -non-internalizing IgG, scFv, Fab, SIP or diabody.

18. The targeted therapeutic agent of claim 7, wherein the tubulin disruptor is a maytansinoid.

19. The targeted therapeutic agent of claim 18, wherein the maytansinoid is selected from DM1, DM3, and DM4.

20. The targeted therapeutic agent of claim 14, wherein the neoplastic disease is a tumor.


SEQUENCE LISTING (15 sequences)

SEQ ID NO: 1 - protein, 121 aa, Artificial Sequence
  Humanized 1959 VH
QVQLQESGPGLVKPSETLSLTCAVSGYSISSGYYWTWIRQPPGKGLEWIGYITYDGKNNYSPSLKNRVTISVDTSKNQFSLKLSSVTAADTAVYYCAREGSSVITTGFTFWGQGTLVTVSS

SEQ ID NO: 2 - protein, 106 aa, Artificial Sequence
  Humanized 1959 VL
DIQMTQSPSTLSASVGDRVTITCSASSRVSYMYWYQQKPGKAPKLLIYLTSNLASGVPARFSGSGSGTEFTLTISSLQPDDFATYYCQQWTSHPPTFGQGTKVEVK

SEQ ID NO: 3 - protein, 11 aa, Artificial Sequence
  Heavy Chain CDR1
GYSISSGYYWT

SEQ ID NO: 4 - protein, 16 aa, Artificial Sequence
  Heavy Chain CDR2
YITYDGKNNYSPSLKN

SEQ ID NO: 5 - protein, 12 aa, Artificial Sequence
  Heavy Chain CDR3
EGSSVITTGFTF

SEQ ID NO: 6 - protein, 10 aa, Artificial Sequence
  Light Chain CDR1
SASSRVSYMY

SEQ ID NO: 7 - protein, 7 aa, Artificial Sequence
  Light Chain CDR2
LTSNLAS

SEQ ID NO: 8 - protein, 9 aa, Artificial Sequence
  Light Chain CDR3
QQWTSHPPT

SEQ ID NO: 9 - protein, 470 aa, Artificial Sequence
  Humanized 1959 HC (wt)
MNFGLRLIFLVLTLKGVQCQVQLQESGPGLVKPSETLSLTCAVSGYSISSGYYWTWIRQPPGKGLEWIGYITYDGKNNYSPSLKNRVTISVDTSKNQFSLKLSSVTAADTAVYYCAREGSSVITTGFTFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 232 aa, Artificial Sequence
  Humanized 1959 LC
MNFGLRLIFLVLTLKGVQCDIQMTQSPSTLSASVGDRVTITCSASSRVSYMYWYQQKPGKAPKLLIYLTSNLASGVPARFSGSGSGTEFTLTISSLQPDDFATYYCQQWTSHPPTFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 451 aa, Artificial Sequence
  Human 1959sss  HC (C220S  C226S  C229S)
QVQLQESGPGLVKPSETLSLTCAVSGYSISSGYYWTWIRQPPGKGLEWIGYITYDGKNNYSPSLKNRVTISVDTSKNQFSLKLSSVTAADTAVYYCAREGSSVITTGFTFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 20 bp, Artificial Sequence
  Primer LGALS3BP Fw
gaacccaaggcgtgaacgat

SEQ ID NO: 13 - DNA, 20 bp, Artificial Sequence
  Primer LGALS3BP Rw
gtcccacaggttgtcacaca

SEQ ID NO: 14 - DNA, 24 bp, Artificial Sequence
  Primer Act Fw
cagctcaccatggatgatgatatc

SEQ ID NO: 15 - DNA, 17 bp, Artificial Sequence
  Primer Act Rw
aagccggccttgcacat